SEQUENCE LISTING (37 sequences)

SEQ ID NO: 1 - DNA, 47 bp, Artificial Sequence
  Synthetically generated oligonucleotide
aattaatacgactcactatagggaaggcctacaaatcggaactggag

SEQ ID NO: 2 - DNA, 22 bp, Artificial Sequence
  Synthetically generated oligonucleotide
gaacaactgaccccggtggcgg

SEQ ID NO: 3 - DNA, 20 bp, Artificial Sequence
  Synthetically generated oligonucleotide
gaggcgaggcgcacccgcag

SEQ ID NO: 4 - DNA, 21 bp, Artificial Sequence
  Synthetically generated oligonucleotide
ttaatacgactcactataggg

SEQ ID NO: 5 - DNA, 46 bp, Artificial Sequence
  Synthetically generated oligonucleotide
cattaatacgactcactatagggactcggggtcgggcttggggaga

SEQ ID NO: 6 - DNA, 49 bp, Artificial Sequence
  Synthetically generated oligonucleotide
cattaatacgactcactatagggacccgggagaggaagatggaattttc

SEQ ID NO: 7 - DNA, 48 bp, Artificial Sequence
  Synthetically generated oligonucleotide
cattaatacgactcactatagggacccgagctgcgccagcagaccgag

SEQ ID NO: 8 - DNA, 48 bp, Artificial Sequence
  Synthetically generated oligonucleotide
cattaatacgactcactatagggacattgcaggcagatagtgaatacc

SEQ ID NO: 9 - DNA, 43 bp, Artificial Sequence
  Synthetically generated oligonucleotide
cattaatacgactcactatagggaaggcctggggcgagcggct

SEQ ID NO: 10 - DNA, 48 bp, Artificial Sequence
  Synthetically generated oligonucleotide
cattaatacgactcactatagggaaggccttccaggcccgcctcaaga

SEQ ID NO: 11 - DNA, 22 bp, Artificial Sequence
  Synthetically generated oligonucleotide
ctcggggtcgggcttggggaga

SEQ ID NO: 12 - DNA, 25 bp, Artificial Sequence
  Synthetically generated oligonucleotide
cccgggagaggaagatggaattttc

SEQ ID NO: 13 - DNA, 24 bp, Artificial Sequence
  Synthetically generated oligonucleotide
cccgagctgcgccagcagaccgag

SEQ ID NO: 14 - DNA, 24 bp, Artificial Sequence
  Synthetically generated oligonucleotide
cattgcaggcagatagtgaatacc

SEQ ID NO: 15 - DNA, 19 bp, Artificial Sequence
  Synthetically generated oligonucleotide
aggcctggggcgagcggct

SEQ ID NO: 16 - DNA, 21 bp, Artificial Sequence
  Synthetically generated oligonucleotide
ccttccaggcccgcctcaaga

SEQ ID NO: 17 - DNA, 22 bp, Artificial Sequence
  Synthetically generated oligonucleotide
cccagtaggtgctcgataaatg

SEQ ID NO: 18 - DNA, 22 bp, Artificial Sequence
  Synthetically generated oligonucleotide
agaagagggggcccagggtctg

SEQ ID NO: 19 - DNA, 24 bp, Artificial Sequence
  Synthetically generated oligonucleotide
tgagtcagaagggaagagagagag

SEQ ID NO: 20 - DNA, 22 bp, Artificial Sequence
  Synthetically generated oligonucleotide
agcacaggtgtgtggcaccatg

SEQ ID NO: 21 - DNA, 21 bp, Artificial Sequence
  Synthetically generated oligonucleotide
ctcgtccaggcggtcgcgggt

SEQ ID NO: 22 - DNA, 21 bp, Artificial Sequence
  Synthetically generated oligonucleotide
tccaccccaggaggacggctg

SEQ ID NO: 23 - DNA, 19 bp, Artificial Sequence
  Synthetically generated oligonucleotide
taatacgactcactatagg

SEQ ID NO: 24 - DNA, 19 bp, Artificial Sequence
  Synthetically generated oligonucleotide
aattaaccctcactaaagg

SEQ ID NO: 25 - DNA, 19 bp, Artificial Sequence
  Synthetically generated oligonucleotide
atttaggtgacactataga

SEQ ID NO: 26 - DNA, 39 bp, Artificial Sequence
  Synthetically generated oligonucleotide
ttaatacgactcactatagggtttttttttttttttttv

SEQ ID NO: 27 - DNA, 33 bp, Artificial Sequence
  Synthetically generated oligonucleotide
gcgccaattatcgaaaaaaaaaaaaaaaaaaaa

SEQ ID NO: 28 - DNA, 58 bp, Artificial Sequence
  Synthetically generated oligonucleotide
ataggcgcgccaattaatacgactcactatagggagattttttttttttttttttttv

SEQ ID NO: 29 - DNA, 58 bp, Artificial Sequence; identical to SEQ ID NO: 28
  Synthetically generated oligonucleotide

SEQ ID NO: 30 - DNA, 71 bp, Artificial Sequence
  Synthetically generated oligonucleotide
acgtacgtacgtcataggcgcgccaattaatacgactcactatagggagattttttttttttttttttttv

SEQ ID NO: 31 - DNA, 96 bp, Artificial Sequence
  Synthetically generated oligonucleotide
acgtacgtacgtacgtacgtacgtcacgtacgtacgtcataggcgcgccaattaatacgactcactatagggagattttttttttttttttttttv

SEQ ID NO: 32 - DNA, 33 bp, Artificial Sequence; identical to SEQ ID NO: 27
  Synthetically generated oligonucleotide

SEQ ID NO: 33 - DNA, 46 bp, Artificial Sequence
  Synthetically generated oligonucleotide
attaatacgactcactatagggagattttttttttttttttttttv

SEQ ID NO: 34 - DNA, 52 bp, Artificial Sequence
  Synthetically generated oligonucleotide
gcgccaattaatacgactcactatagggagattttttttttttttttttttv

SEQ ID NO: 35 - DNA, 58 bp, Artificial Sequence; identical to SEQ ID NO: 28
  Synthetically generated oligonucleotide

SEQ ID NO: 36 - DNA, 41 bp, Artificial Sequence
  Synthetically generated oligonucleotide
taataggttgtattgatgttggacgagtcggaatcgcagac

SEQ ID NO: 37 - DNA, 30 bp, Artificial Sequence
  Syntheticaly generated oligonucleotide
ttgccatcctatggaactgcctcggtgagt

(19) United States
(12) Patent Application Publication
(10) Pub. No.: US 2005/0064432 A1
(43) Pub. Date: Mar. 24, 2005

(54) Nucleic acid amplification and detection

(75) Inventors: Tai-Nang Huang, Lexington, MA (US); Simon W. Law, Lexington, MA (US); Haisun Liao, Sharon, MA (US)

Correspondence Address: FISH & RICHARDSON PC, 225 FRANKLIN ST, BOSTON, MA, 02110, US

(73) Assignee: Linden Technologies, Inc.

(21) Appl. No.: 10/664,608

(22) Filed: Sep. 19, 2003

(51) Int. Cl. C12Q 1/68; C12M 1/34

(52) U.S. Cl. 435/6; 435/287.2

(57) ABSTRACT

Disclosed is a substrate that includes a promoter primer that can be extended to form a transcribable template nucleic acid; and a capture probe. Typically, the promoter primer and the capture probe are non-complementary, and the capture probe can specifically bind to a target nucleic acid. The substrate can be used to amplify and detect one or more target nucleic acids.

### BACKGROUND

Genetic information can be analyzed for a number of applications, including medical diagnosis, genotyping, and forensics. The high throughput analysis of nucleic acid samples is facilitated by nucleic acid amplification. A variety of techniques can be used for nucleic acid amplification. The polymerase chain reaction (PCR; Saiki, et al. (1985) Science 230, 1350-1354) and ligase chain reaction (LCR; Wu. et al. (1989) Genomics 4, 560-569; Barringer et al. (1990), Gene 1989, 117-122; F. Barany. 1991, Proc. Natl. Acad. Sci. USA 1988, 189-193) utilize cycles of varying temperature to drive rounds of synthesis. Transcription-based methods utilize RNA synthesis by RNA polymerases to amplify nucleic acid (U.S. Pat. Nos. 6,066,457, 6,132,997, and 5,716,785; Sarkar et al., Science (1989) 244:331-34; Stofler et al., Science (1988) 239:491). NASBA (U.S. Pat. Nos. 5,130,238; 5,409,818; and 5,554,517) utilizes cycles of transcription, reverse-transcription, and RNaseH-based degradation to amplify a DNA sample. Still other amplification methods include rolling circle amplification (RCA; U.S. Pat. Nos. 5,854,033 and 6,143,495) and strand displacement amplification (SDA; U.S. Pat. Nos. 5,455,166 and 5,624,825). There is need for simple devices that can detect nucleic acids. Some embodiments of the present invention address this need as well as other problems.

### SUMMARY

In one aspect, the invention features a substrate that includes: (a) a promoter primer that can be extended to form a transcribable template nucleic acid; and (b) a capture probe, wherein the promoter primer and the capture probe are non-complementary, and the capture probe can specifically bind to a target nucleic acid or complement thereof. In one embodiment, the capture probe is exactly complementary to the target nucleic acid. For example, the capture probe include a region of between 10-1000, 10-100, or 14-30 nucleotides in length that is exactly complementary to the target nucleic acid. In one embodiment in which the substrate is used for amplification, depending on whether the target nucleic acid is amplified as a sense or anti-sense strand, the capture probe can be designed to have the correct complementarity.

In one embodiment, the capture probe is un-extendable. For example, the capture probe can be blocked at its '3 end. In one embodiment, the capture probe is attached (e.g., covalently or non-covalently) to the substrate by its 3′ end. In one embodiment, the 3′ end of the capture probe lacks a terminal hydroxyl.

In one embodiment, the promoter primer includes a polyT sequence that can anneal to the 3′ end of eukaryotic mRNAs. For example, the polyT sequence terminates with a mono or dinucleotide other than T at the 3′ most region of the promoter primer. In another example, it terminates in T.

In one embodiment, the promoter primer includes a target specific sequence that can anneal to a target RNA or DNA sequence, e.g., a mRNA or rRNA or a genomic DNA or a mitochondrial DNA. For example, the target sequence can be from a pathogen, e.g., a bacterial, protist, fungal, or nematode pathogen. The pathogen can be a pathogen to a human, an animal (e.g., a domesticated animal, e.g., an agricultural animal or a pet), or to plants (e.g., a cultivated, agricultural plant). In one embodiment, the promoter primer includes a sequence that is recognized by a prokaryotic RNA polymerase to initiate transcription, e.g., a T3 or T7 promoter. In one embodiment, the capture probe can bind to a different region (e.g., a non-overlapping or less than 50% overlapping) of the target nucleic acid than the target specific sequence that is recognized by the promoter primer.

In one embodiment, the substrate is planar. In one embodiment, the substrate includes a microchannel that places a region of the substrate occupied by the promoter primer in fluid communication with a region occupied by the capture probe. For example, the microchannel is etched. The substrate can include other features described herein.

In another aspect, the invention features a method that includes providing a substrate described herein; contacting a sample to the substrate; forming template nucleic acids by extending the promoter primer; transcribing the template nucleic acids, thereby providing transcripts; and evaluating binding of the transcripts to the capture probe.

In one embodiment, the promoter is a prokaryotic promoter.

In one embodiment, the transcribing is effected in the presence of a labeled ribonucleotide that can be incorporated into the transcripts. In one embodiment, the evaluating includes detecting presence of the labeled ribonucleotides incorporated into the transcripts that bind to the capture probe. In one embodiment, the evaluating includes contacting a detector probe to the substrate, wherein the detector probe can specifically anneal to a region of the target nucleic acid that does not overlap with the capture probe. For example, the detector probe includes a label, e.g., a light-detectable label, e.g., a fluorophore, a tag or enzyme.

In another aspect, the invention features a substrate that includes: immobilized template nucleic acids that include a promoter region; one or more RNA polymerases that can collectively transcribe the immobilized template nucleic acids, and a capture probe that can hybridize to a target nucleic acid, if present. The substrate can further include a detector probe,

In one embodiment, the capture probe is immobilized, but the detector probe is present in a diffusable state on the substrate. In one embodiment, the substrate is dry, and the RNA polymerases are provided in a lyophilized state on the substrate. The lyophilized material provides active polymerase enzyme upon hydration. In one embodiment, the RNA polymerases are provided in a crystalline form. In one embodiment, the substrate includes an aqueous coat that includes a cryoprotectant. The substrate can be cooled to below 4° C. The substrate can include other features described herein.

In another aspect, the invention features a substrate that includes a plurality of regions. Each region includes (a) a promoter primer that can be extended to form a transcribable template nucleic acid, and (b) a first capture probe. In one embodiment, the first capture probe of each region is non-extendable. In one embodiment, the first capture probe and the promoter primer do not interact. In one embodiment, the promoter primer cannot be extended. In one embodiment, the promoter primer and the first capture probe are both covalently coupled to the substrate.

In one embodiment, each region further includes a second capture probe. For example, the first and second capture probe can detect different cellular nucleic acids, e.g., they are complementary to different cellular nucleic acids.

In one embodiment, the first and second capture probes recognize different regions of a common cellular nucleic acid, and the plurality of regions includes regions for different cellular nucleic acids. The substrate can include other features described herein.

In another aspect, the invention features a nucleic acid detection kit. The kit includes a substrate described herein. The kit can further include instruction for use, e.g., for a diagnostic. The kit can further include one or more detector nucleic acids, e.g., a labeled detector nucleic acid. The kit can further include one or more reagents for labelling the detector nucleic acid.

The kit can further include an agent that transcribes the promoter primer. The kit can further include reagents for forming a transcribable template nucleic acid from the promoter primer. The kit can include other features described herein.

In another aspect, the invention features a device that includes: (a) means for forming transcribable templates for a plurality of different nucleic acids, (b) means for capturing specific nucleic acids, and (c) means for supporting (a) and (b). The device can include other features described herein.

In another aspect, the invention features a method that includes: contacting a sample to a substrate that includes an immobilized promoter primer, wherein the promoter primer includes a sequence that forms at least one strand of a transcription recruitment sequence and an annealing sequence; producing a transcribable nucleic acid template by extended the promoter primer, if the sample contains a sequence that can hybridize to the annealing sequence; transcribing the transcribable nucleic to produce transcripts; and evaluating interaction between transcripts and a capture probe without removing the transcripts from the substrate.

In one embodiment, the capture probe is immobilized on the substrate, e.g., prior to contacting the sample to the substrate. In one embodiment, the promoter primer and the capture probe are within a diffusable distance of each other. In one embodiment, the promoter primer is immobilized on the substrate prior to contacting the sample to the substrate.

In one embodiment, the evaluating includes hybridizing detector probe(s) to the transcripts and washing detector probe(s) that are not attached to the substrate in a complex that includes the capture probe and the transcript from the substrate. For example, the detector probe includes a label, e.g., a light-detectable label, e.g., a fluorescent label

In one embodiment, the sample includes less than 100, 10, 1, 0.1, or 0.01 pg of the target nucleic acid.

In another aspect, the invention features a method that includes: providing a substrate that includes a plurality of regions; and modifying the substrate so that each region includes
(a) a promoter primer that can be extended to form a transcribable template nucleic acid, and (b) a capture probe. In one embodiment, the modifying includes synthesizing the promoter primer and/or the capture probe on the substrate. In one embodiment, the modifying includes disposing the promoter primer and the capture probe on the substrate. For example, the modifying includes disposing the promoter primer and the capture probe at the same time. or separately. In one embodiment, the promoter primer is disposed in one subregion of the region, and the capture probe is disposed in another subregion of the region.

In one embodiment, the substrate includes demarcations that physically separate the regions of the plurality from each other. For example, the demarcations are optically detectable. The demarcations can form an impermeable barrier, e.g., a solid barrier.

In one embodiment, the impermeable barrier and the substrate are composed of the same material and are rigidly connected. The substrate can include other features described herein.

One object of some embodiments of the nucleic acid amplification and detection methods described herein is to provide an expedient method that requires little participation and manipulation by a user. Another object of some embodiments is to provide a single device which can perform both nucleic acid amplification and detection.

In one embodiment, the methods can be integrated to provide a single platform that can perform both template formation, transcription, and detection of a potential target. The single platform can be provided, for example, by providing a substrate that includes both the promoter primer and capture probe. Using this substrate, amplified nucleic acids can be captured as they are produced, thus obviating the need to transfer and hybridize the amplified nucleic acids.

In one embodiment, the device includes a microfluidic channel. For example, the channel enables fluid communication between a region of the substrate that includes the promoter primer and a region of the substrate that includes the capture probe. The application of microfluidics for reagent supply simplifies the automation design.

One embodiment uses three different nucleic acids. For example, two of these nucleic acids (e.g., the capture probe and the detector probe) can be specific. In addition, it is also possible to use a specific promoter primer, e.g., by using a sequence specific target-annealling sequence. The use of at least two specific nucleic acids can aid accuracy.

The methods and arrays described here can be used for transcription amplification. Exemplary applications include evaluate genomic nucleic acid (e.g., for a polymorphism), evaluating one or more transcripts (e.g., to evaluate a state of a cell or tissue). Non-limiting implementations include medical diagnostics, forensics, disease gene mapping, environmental management, agriculture, and protein evolution. The methods can be used to evaluate samples that include very low concentrations of nucleic acid, e.g., evaluating transcripts in a sample, e.g., a sample that includes cells, e.g., fewer than 10 000 or 1 000 cells.

In another aspect, the invention features a method that includes: providing a substrate including promoter primers that include a promoter sequence and a target annealing sequence, annealing sample nucleic acids to the promoter primers; constructing template nucleic acids by extending the promoter primers using a polymerase; and transcribing the template nucleic acids to produce RNA replicates of the sample nucleic acids. In one embodiment, the substrate further includes a capture probe, and the method can further include detecting interaction between one or more of the RNA replicates and a capture probe.

In one embodiment, the proximal end of the promoter sequence is spaced from the substrate by a distance greater than 5 nm or 10 nm. For example, the distance can be between 10 to 150 nm or 10 and 50 nm. In one embodiment, the promoter primers each include a ligand 5′ of the promoter (e.g., at the 5′ terminus), are attached to the substrate by a non-covalent interaction (e.g., a ligand/ligand-binding protein interaction), and the proximal end of the promoter is between 5-30, 6-18, or 6-12 nucleotides from the ligand, e.g., from the 5′ terminus. For example, the promoter primers include a biotin moiety (e.g., at the 5′ terminus), are attached to the substrate by a biotin/biotin-binding protein interaction (e.g., avidin or streptavidin), and the proximal end of the promoter is at least 5 nucleotides, e.g., between 5-30 nucleotides from the biotin moiety.

In another embodiment, the promoter primers are attached to the substrate by a polyethylene glycol linker that has at least 8 units or between 8-20 or 8-16 units, by a chemical linker having a main chain length including the same number of main chain atoms as the polyethylene glycol linker or having the same physical length as the polyethylene glycol linker.

In another embodiment, the promoter primers are covalently attached at their 5′ terminus, and the proximal end of the promoter is between 12-50, 20-35, or 23-28 nucleotides from the 5′ terminus of each of the promoter primers.

The sample nucleic acids include RNA, DNA, PNA, or other nucleic acid molecules.

The constructing can include extending the promoter primer using an RNA-directed DNA polymerase to produce an extended stranded and synthesizing a DNA strand complementary to the extended strand to produce complementary strands, e.g., by a method described herein. The attached and complementary strands anneal, thereby providing the template nucleic acids.

The method can include joining an adaptor that includes a tag sequence to the double-stranded template. The adaptor can include a promoter sequence, e.g., a prokaryotic promoter sequence. For example, the adaptor includes double-stranded DNA.

A “promoter” is a sequence at which a polymerase can initiate transcription. The promoter sequence can be a prokaryotic promoter sequence, e.g., a bacteriophage promoter sequence, e.g., a T7, T3, or SP6 promoter sequence. In one embodiment, at least some of the promoter primers include a promoter and a homopolymeric T tract. These promoter primers can further include a 3′ terminal A, G, or C.

The distance between the proximal end of the promoter sequence and the substrate can be sufficient to enable at least 2, 4, 8, or 16 times the yield (e.g., between 2 and 32 times) of replicate RNAs as obtained using a distance of less than 2 nm between the proximal end of the promoter sequence and the substrate.

In another aspect, the invention features a method that includes: providing a substrate including attached template nucleic acids (e.g., covalently or non-covalently attached), wherein (1) each attached template nucleic acid includes a promoter sequence and a target sequence, and (2) the proximal end of the promoter sequence is spaced from the substrate by a predetermined distance; and transcribing the template nucleic acids to produce RNA replicates of the sample nucleic acids. For example, the distance between the proximal end of the promoter sequence and substrate can be between 10 to 150 nm or 10 and 50 nm.

In one embodiment, the attached template nucleic acids each include a ligand 5′ of the promoter (e.g., at the 5′ terminus), are attached to the substrate by a non-covalent interaction (e.g., a ligand/ligand-binding protein interaction), and the proximal end of the promoter is between 5-30, 6-18, or 6-12 nucleotides from the ligand, e.g., from the 5′ terminus. For example, the attached templates include a biotin moiety (e.g., at the 5′ terminus), are attached to the substrate by a biotin/biotin-binding protein interaction (e.g., avidin or streptavidin), and the proximal end of the promoter is at least 5 nucleotides, e.g., between 5-30 nucleotides from the biotin moiety.

In another embodiment, the attached template nucleic acids are attached to the substrate by a polyethylene glycol linker that has at least 8 units or between 8-20 or 8-16 units, by a chemical linker having a main chain length including the same number of main chain atoms as the polyethylene glycol linker or having the same physical length as the polyethylene glycol linker.

In another embodiment, the attached template nucleic acids are covalently attached at their 5′ terminus, and the proximal end of the promoter is between 12-50, 20-35, or 23-28 nucleotides from the 5′ terminus of each of the attached template nucleic acids.

The template nucleic acids can further include a second promoter positioned to transcribe a nucleic acid segment located between the first and second promoters. Each is configured to transcribe a strand of the nucleic acid segment such that both strands of the nucleic acid segment are transcribed. This method includes transcribing the template nucleic acid using the first and second promoters to produce RNA complementary to each strand, and recovering double-stranded RNA for the nucleic acid segment.

The template nucleic acids can correspond to nucleic acids in a biological sample, e.g., a sample of nucleic acids obtained from a cell, e.g., from a culture cell, tissue, free-living cell or organism. The template nucleic acids can include regions that represent nucleic acids in the sample in comparable proportions. For example, the template nucleic acids can correspond to eukaryotic mRNAs, genomic DNAs, and so forth.

In one embodiment, a plurality of the template nucleic acids each comprises a common adaptor sequence at their respective distal ends. The adaptor sequence can include a promoter sequence.

In another aspect, the invention features a method of archiving a sample of complex nucleic acids. The method includes: providing a first substrate having 5′ promoter primer, wherein the promoter primer includes a promoter sequence that is at least 4 nm from the substrate; annealing a complex sample that includes sample nucleic acids to the substrate; and producing template nucleic acids immobilized on the substrate that each include at least a segment of the sample nucleic acids, the immobilized templates representing the composition of the sample nucleic acids; transcribing the template nucleic acids from the substrate; archiving the substrate; and transcribing the template nucleic acids from the substrate. For example, the distance can be between 10 to 150 nm or 10 and 50 nm.

In one embodiment, the promoter primers each include a ligand 5′ of the promoter (e.g., at the 5′ terminus), are attached to the substrate by a non-covalent interaction (e.g., a ligand/ligand-binding protein interaction), and the proximal end of the promoter is between 5-30, 6-18, or 6-12 nucleotides from the ligand, e.g., from the 5′ terminus. For example, the promoter primers include a biotin moiety (e.g., at the 5′ terminus), are attached to the substrate by a biotin/biotin-binding protein interaction (e.g., avidin or streptavidin), and the proximal end of the promoter is at least 5 nucleotides, e.g., between 5-30 nucleotides from the biotin moiety.

In another embodiment, the promoter primers are attached to the substrate by a polyethylene glycol linker that has at least 8 units or between 8-20 or 8-16 units, by a chemical linker having a main chain length including the same number of main chain atoms as the polyethylene glycol linker or having the same physical length as the polyethylene glycol linker.

In another embodiment, the promoter primers are covalently attached at their 5′ terminus, and the proximal end of the promoter is between 12-50, 20-35, or 23-28 nucleotides from the 5′ terminus of each of the promoter primers.

The template nucleic acids can correspond to nucleic acids in a biological sample, e.g., a sample of nucleic acids obtained from a cell, e.g., from a culture cell, tissue, free-living cell, subcellular and remnant particles, or organism. The template nucleic acids can include regions that represent nucleic acids in the sample in comparable proportions. For example, the template nucleic acids can correspond to eukaryotic mRNAs, genomic DNAs, and so forth.

The invention also features substrates and immobilized oligonucleotides described herein, e.g., including one or more features described herein. The substrates can include template nucleic acids, e.g., including features acquired from a sample described herein.

In another aspect, the invention features a substrate that includes a plurality of promoter primers, wherein (1) the promoter primers include a prokaryotic promoter sequence and a target annealing sequence, (2) the target annealing sequence is 3′ of the promoter, (3) the oligonucleotide has an extendable 3′ terminus; and (4) the proximal end of the promoter sequence is spaced from the substrate by a distance greater than 10 nm,

In one embodiment, the oligonucleotides are less than 80 nucleotides in length.

In one embodiment, each target annealing sequence of the plurality is the same and the target annealing sequence can anneal to a plurality of different target sequences. For example, the target annealing sequence can include a poly-thymidine tract. Each target annealing sequence of the plurality can include a poly-thymidine tract and a terminal 3′ A, G, or C.

In one embodiment, target nucleic acids are annealed to the substrate, e.g., target nucleic acids from a sample described herein.

In one embodiment, the promoter primers each include a ligand 5′ of the promoter (e.g., at the 5′ terminus), are attached to the substrate by a non-covalent interaction (e.g., a ligand/ligand-binding protein interaction), and the proximal end of the promoter is between 5-30, 6-18, or 6-12 nucleotides from the ligand, e.g., from the 5′ terminus. For example, the promoter primers include a biotin moiety (e.g., at the 5′ terminus), are attached to the substrate by a biotin/biotin-binding protein interaction (e.g., avidin or streptavidin), and the proximal end of the promoter is at least 5 nucleotides, e.g., between 5-30 nucleotides from the biotin moiety.

In another embodiment, the promoter primers are attached to the substrate by a polyethylene glycol linker that has at least 8 units or between 8-20 or 8-16 units, by a chemical linker having a main chain length including the same number of main chain atoms as the polyethylene glycol linker or having the same physical length as the polyethylene glycol linker.

In another embodiment, the promoter primers are covalently attached at their 5′ terminus, and the proximal end of the promoter is between 12-50, 20-35, or 23-28 nucleotides from the 5′ terminus of each of the promoter primers.

In another aspect, the invention features a substrate that includes attached template nucleic acids, wherein (1) each attached template nucleic acids include a prokaryotic promoter sequence, a target sequence, and a ligand (2) for each template nucleic acid, the promoter is located between the target sequence and ligand, (3) the template nucleic acids can be transcribed to produce RNA copies of each respective target sequence, (4) the ligand is bound to a ligand-binding protein immobilized on the substrate, and (5) the proximal end of the promoter sequence is spaced from the ligand between 5 and 30 nucleotides.

In a related aspect, the substrate includes attached template nucleic acids, wherein (1) each attached template nucleic acids include a prokaryotic promoter sequence and a target sequence, (2) for each template nucleic acid, the promoter is located between the target sequence and the site that attaches the template nucleic acid to the substrate, (3) the template nucleic acids can be transcribed to produce RNA copies of each respective target sequence, and (4) the template nucleic acids is spaced from the substrate by a nucleotide-free linker that includes an identical number of main chain atoms as a polyethylene glycol linker that has at least 8 units or between 8 and 16 units.

In another related aspect, the substrate includes attached template nucleic acids, wherein (1) each attached template nucleic acids include a prokaryotic promoter sequence and a target sequence, (2) for each template nucleic acid, the promoter is located between the target sequence and the site that attaches the template nucleic acid to the substrate, (3) the template nucleic acids can be transcribed to produce RNA copies of each respective target sequence, and (4) the attached template nucleic acids are covalently attached to the substrate, and the proximal end of the promoter is between 12 and 50 nucleotides from the 5′ terminus of each of the oligonucleotides.

The template nucleic acids can correspond to nucleic acids in a biological sample, e.g., a sample of nucleic acids obtained from a cell, e.g., from a culture cell, tissue, free-living cell, subcellular and remnant particles, or organism. The template nucleic acids can include regions that represent nucleic acids in the sample in comparable proportions. For example, the template nucleic acids can correspond to eukaryotic mRNAs, genomic DNAs, and so forth.

In one embodiment, a plurality of the template nucleic acids each comprises a common adaptor sequence at their respective distal ends. The adaptor sequence can include a promoter sequence.

In another aspect, the invention features a method that includes: cleaving sample nucleic acids to yield cleaved nucleic acids; treating the cleaved nucleic acids using a nuclease that preferentially digests double stranded nucleic acid relative to single stranded nucleic acid to yield treated sample nucleic acids; annealing a promoter primer to the treated sample nucleic acids, wherein the promoter primer (also referred to as the “SSP oligonucleotide”) includes a promoter region and a target binding region that binds to a first target site; and transcribing the annealed treated sample nucleic acid using an RNA polymerase that recognizes the promoter region to generate RNA replicates of the sample nucleic acid. The promoter primer can include an element that spaces the promoter from the substrate, e.g., by a distance described herein. The method is useful for amplifying sample nucleic acid.

In one embodiment, the method further includes, prior to or concurrent with the transcribing, extending the promoter primer and/or the annealed sample nucleic acid using a DNA polymerase. The DNA polymerase can lack 3′ to 5′ exonucleases activity. For example, the DNA polymerase can be the Klenow fragment of E. coli DNA polymerase I, or a modified or unmodified bacteriophage DNA polymerase such as SEQUENASE™. In one embodiment, the method includes separating the extended strands from the unannealed and/or unextended sample nucleic acid strands prior to transcription. In another embodiment, only the annealed sample nucleic acid is extended, i.e., thereby rendering the promoter region double stranded and functional. The promoter primer can have a 3′ modification that prevents its extension.

In one embodiment, the promoter primer includes a moiety that is attachable to receiving agent. The receiving agent can be attached to a substrate, e.g., a bead or planar surface. In one embodiment, the moiety and receiving agent are members of a specific binding pair, e.g., biotin and avidin (or streptavidin), sugar and lectin, and so forth. In another embodiment, the moiety and receiving agent are chemically reactive with each other. For example, the moiety can be an amino group and the receiving agent can be an activated group that includes an electron-withdrawing group on an N-substituted sulfonamide.

The method can be performed at temperatures of less than about 50, 45, or 40° C. In other words, in some implementations, the reaction temperature never exceeds these temperatures. The method can be performed under isothermal or substantially isothermal conditions. Further enzymes used in one or more reactions can be added and removed by flowing or otherwise altering the medium that contacts the substrate. In one embodiment, pins or other devices that include the SSP oligonucleotide immobilized thereto can be moved from one reaction mixture to another.

The cleaving can include shearing, sonication, or digestion using a cleaving agent such as an endonuclease, e.g., one or more restriction endonucleases. The restriction endonucleases can specifically recognize a 4, 5, or 6 base pair site. They can digest DNA to produce recessed ends, e.g., 5′ overhangs, or blunt ends. The sample nucleic acid can be, for example, DNA or RNA. In a preferred embodiment, the sample nucleic acid is DNA, e.g., genomic DNA, cDNA, or recombinant DNA.

The cleaving can generate fragments having an average size of less than about 2000, 1000, 700, or 500 nucleotides or can generate a fragment in a region of interest of less than about 2000, 1000, 700, or 500 nucleotides. The method can include inactivating the cleaving agent and/or separating the cleaved nucleic acids from the cleaving agent.

The nuclease that is used to treat the cleaved nucleic acid preferentially digests double stranded nucleic acid relative to single stranded nucleic acid. A preferential digestion as used herein, refers to at least a 50-fold difference in Kₘ for the respective substrates. The nuclease can be highly processive. The nuclease can be an exonuclease, e.g., lambda exonuclease or T7 exonuclease.

The nuclease can be attached to a substrate, e.g., a bead, such as a paramagnetic bead. The method can further include separating the nuclease from the treated sample nucleic acids. The method can include inactivating the nuclease.

The method can further include reverse transcribing the RNA replicates and/or treating the RNA replicates using a ribonuclease, e.g., RNaseH. In another embodiment, the method can further include translating the RNA replicates. In still another embodiment, the method can further include analyzing the RNA replicates or DNA copies thereof. The analysis can include determining the identity of a nucleotide or the sequence of a region. The analysis can indicate whether an allele or polymorphism is present.

In another aspect, the invention features a method that includes: providing a substrate having a plurality of addresses; and at each of the plurality of addresses, providing (e.g., depositing or synthesizing) a promoter primer and a capture probe. The promoter primer can include a 5′ promoter region and a 3′ target binding region that is complementary to a target site. The method can further include contacting a sample of nucleic acid to the substrate; for each of the promoter primers of the plurality of addresses, permitting the target binding region to anneal to its target site in the sample, if present; extending the annealed sample nucleic acid using a DNA polymerase (e.g., thereby rendering the promoter region of the oligonucleotide double-stranded); and transcribing the annealed sample nucleic acid using an RNA polymerase that recognizes the promoter region. The promoter primer can include an element that spaces the promoter from the substrate, e.g., by a distance described herein.

In one embodiment, the promoter regions are the same among the promoter primers of the plurality of addresses. In another embodiment, the promoter regions are different.

The method can include, prior to the extending or the transcribing, separating unannealed sample nucleic acids or separate annealed and unannealed sample nucleic acids (e.g., after extending the annealed promoter primers to copy).

In one embodiment, the promoter primer is extended using a DNA polymerase.

In another embodiment, the substrate is positioned in a flow chamber. The RNA polymerase and ribonucleotides are provided to the chamber as transcription products are removed from the chamber.

In still another aspect, the invention provides a method that includes: providing a substrate having a plurality of addresses, each address including (1) a first nucleic acid segment having (a) a 5′ promoter region and (b) a variable 3′ target binding region, and (2) a second nucleic acid segment that binds the 5′ promoter region; annealing sample nucleic acids to the substrate; joining the 5′ terminus of the second nucleic acid segment to the 3′ end of the annealed sample nucleic acid; optionally removing unjoined and/or unannealed sample nucleic acids; and transcribing the joined sample nucleic acids using an RNA polymerase that recognizes the 5′ promoter region. The first nucleic acid segment can include an element that spaces the promoter from the substrate, e.g., by a distance described herein.

In one embodiment, the first nucleic acid segment and the second nucleic acid segment are segments of a single nucleic acid strand, e.g., a hairpin strand. The hairpin can include a modified nucleotide or backbone position in the hairpin loop. The modification includes a moiety that is attached to the substrate. The position of the hairpin can be selected such that the 5′ end of the promoter region is spaced from the substrate, e.g., by a distance described herein.

The joining can be effected by a ligase, e.g., T4 DNA ligase, or a thermostable ligase. A thermostable ligase can be useful for annealing at temperatures above 40° C. in order to increase annealing specificity.

In one embodiment, the method includes storing or archiving the substrate. The substrate can be stored any time after the joining of the annealed sample nucleic acid, e.g., prior to the transcribing, or after the transcribing.

In another aspect, the invention provides a method of analyzing genetic polymorphisms. The method includes: for each polymorphism, locating a fragment flanked by restriction enzyme sites and including the polymorphism such that the sites are less than about 2000, 1000, 700, 500 nucleotides apart; synthesizing a promoter oligonucleotide having (a) a 5′ promoter region and (b) a variable 3′ target binding region, the variable 3′ target binding region being near or flanking one of fragment termini; optionally attaching the promoter oligonucleotide to a substrate; annealing sample nucleic acid to the promoter oligonucleotides; contacting a DNA polymerase to the annealed sample nucleic acids to extend the annealed sample nucleic acid and render the promoter double-stranded; and transcribing the extended annealed sample nucleic acid using an RNA polymerase specific for the promoter.

In another aspect, the invention provides a method of analyzing genetic polymorphisms. The method includes: for each polymorphism, synthesizing a promoter oligonucleotide on a substrate, the promoter oligonucleotide having (a) a 5′ terminus attached to the substrate; (b) a 5′ promoter region and (c) a variable 3′ target binding region, the variable 3′ target binding region being within 1000 nucleotides (e.g., less than 800, 700, 500, or 400 nucleotides) of the polymorphism; annealing sample nucleic acid to the promoter oligonucleotides; contacting a DNA polymerase to the annealed sample nucleic acids to extend the annealed sample nucleic acid and render the promoter double-stranded; and transcribing the extended annealed sample nucleic acid using an RNA polymerase specific for the promoter. The promoter oligonucleotide can include an element that spaces the promoter from the substrate, e.g., by a distance described herein.

In another aspect, the invention features a method of amplifying a nucleic acid strand. The method includes: annealing a nucleic acid strand to a first oligonucleotide that binds to the strand; extending the strand 3′ end to form a first oligonucleotide-strand complex; transcribing the first oligonucleotide-strand complex using a first RNA polymerase to yield a first RNA strand; annealing the first RNA to a second oligonucleotide that binds to the first RNA strand; reverse transcribing the first RNA to yield to a first copy strand; rendering the first copy strand double-stranded to form a second oligonucleotide-copy strand complex or annealing a third oligonucleotide that is complementary to the promoter region of the second oligonucleotide; and transcribing the second oligonucleotide-copy strand complex.

The first oligonucleotide includes a promoter region, specifically recognized by a first RNA polymerase, and a target binding region that binds the strand 3′ end. The second oligonucleotide includes a promoter region, specifically recognized by a second RNA polymerase, and a target binding region that binds the first RNA strand 3′ end. The first and second oligonucleotides can bind to their targets near the target 3′ end, e.g., at a location with the strand terminus, or located near the strand terminus within 25% of the length of the strand. The first and/or second oligonucleotide can include a spacer, e.g., that separates the promoter and the substrate attachment site by a distance described herein.

The method can be performed in a homogenous reaction mixture.

In another aspect, the invention features a kit that includes: (1) a prokaryotic RNA polymerase; (2) a DNA polymerase that lacks 3′ to 5′ exonuclease activity; and (3) an exonuclease that is processive and that preferentially digests double stranded nucleic acid relative to single stranded nucleic acid.

The kit can further include: a promoter oligonucleotide that includes (a) a 5′ promoter region that is recognized by the prokaryotic RNA polymerase and (b) a variable 3′ target binding region. In another embodiment, the kit includes a plurality of promoter oligonucleotides. In another embodiment, the kit includes a substrate that is attached to the promoter oligonucleotide or promoter oligonucleotides. The promoter oligonucleotides can include an element that spaces the 5′ end of the promoter form the substrate by a distance described herein.

In another embodiment, the kit further includes ribonucleotides and/or deoxy-ribonucleotides. In yet another embodiment, the kit further includes a container that includes a plurality of restriction endonucleases. The kit can further one or more reaction containers, e.g., microtiter plates, strips, wells, cassettes, and microfluidic devices.

In another aspect, the invention features a pool of non-naturally occurring RNA strands.

The RNA strands are less than about 1000, 700, or 500 nucleotides in length. In one embodiment, at least some or all of the RNA strands have a nucleic acid sequence which is absent from fully processed mRNA. For example, the RNA strands can be transcribed from fragments of genomic DNA which include introns and/or regulatory regions, e.g., transcriptional regulatory regions. The RNA strands can include a common 5′ end, e.g., corresponding to a linker sequence from an SSP oligonucleotide. The common 5′ end can be about 2 to 50 nucleotides in length. The 5′ end can include an internal ribosome entry site, an initiator methionine, and so forth. The RNA can be uncapped.

In still another aspect, the invention features a reaction mixture that includes: (1) a prokaryotic RNA polymerase; and (2) a plurality of oligonucleotides, each oligonucleotide including (a) a 5′ promoter region that is recognized by the prokaryotic RNA polymerase and (b) a variable 3′ target binding region. The mixture can further include: (3) ribonucleotides. In another embodiment, the mixture further includes: (4) a DNA polymerase that lacks 3′ to 5′ exonuclease activity; and (5) deoxyribonucleotides. In one embodiment, the mixture can be used to support a homogeneous reaction in which DNA and RNA are synthesized.

The reaction mixture can further include a second RNA polymerase and a second plurality of oligonucleotides, each of the oligonucleotides including (a) 5′ promoter region that recognized by the second RNA polymerase, and (b) a variable 3′ target binding region.

In one embodiment, the target binding region of the oligonucleotides of the second plurality can bind to a strand complementary to that bound the target binding region of an oligonucleotide of the first plurality. The two respective target binding regions can be within about 4, 2, 1, 0.7, 0.5, 0.3, or 0.1 kb of one another.

In still another aspect, the invention features a substrate that includes a plurality of addresses, each address of the plurality having attached thereto an oligonucleotide that has (a) a 5′ promoter region that is recognized by a prokaryotic RNA polymerase and (b) a variable 3′ target binding region. The variable target binding region can be between about 12 and 50 nucleotides in length. The target binding region can have a Tₘ for annealing to its target of between about 24° C. to 85° C., e.g., about 38° C. to 70° C. The substrate can be a bead, a matrix a planar surface such as a glass slide, membrane, plastic, or a pliable sheet.

In still another aspect, the invention features a substrate that includes a first and second plurality of addresses, each address of the first and second plurality having attached thereto an oligonucleotide that has (a) a 5′ promoter region that is recognized by a prokaryotic RNA polymerase and (b) a variable 3′ target binding region. At each of address of the first plurality, the promoter region of the promoter primer is recognized by a first RNA polymerase. At each address of the second plurality, the promoter region of the promoter primer is recognized by a second RNA polymerase.

In one embodiment, the target binding regions of each of the oligonucleotides of the first plurality binds a target site which is on a strand complementary to the target site bound by a target binding region of an oligonucleotide of the second plurality.

The invention also features methods of using the substrate, e.g., the SP-TCR method.

The invention also features a kit including a first substrate and a second substrate. The first substrate is an array of SSP oligonucleotides. The second substrate is an array of detection probes, each probe querying an allele of a fragment amplifiable by the SSP oligonucleotide array.

In another aspect, the invention features a system that includes: a processor; an array synthesizer; and a repository of polymorphism information. The processor is interfaced with the array synthesizer. The array synthesizer is receives input information that is used to construct an array having 5′ anchored SSP oligonucleotides at each address of a plurality of array addresses. The processor can be configured with software to receive a set of polymorphisms for analysis; lookup or compute an appropriate SSP oligonucleotides; and send instructions to the array synthesizer to synthesize an array having primers for the SSAT amplification of the set polymorphisms or an array of detection primers.

The system can further include an array scanner that is also interfaced with the processor. The array scanner can send results from scanning detection arrays to the processor. The results can be stored in a repository of results.

In yet another aspect, the invention features a method that includes: providing a substrate having promoter primers; annealing a sample that comprises RNAs to the substrate; extending the promoter primers using an RNA-directed DNA polymerase to construct DNA replicates of the RNAs; synthesizing DNA strands complementary to the DNA replicates; and transcribing the complementary strand using an RNA polymerase that recognizes the promoter region to produce RNA replicates. Typically, the RNA replicates are anti-sense with respect to the sample RNAs. The sample RNAs can include RNAs, e.g., obtained from a tissue sample such as a mammalian tissue sample. The sample RNAs can be obtained from less than about 1000, 100, or 10 cells. For example, the sample RNAs can be obtained from about 1, 2, 3, or 5 cells. The mRNA can be is less than 10 ng. In one example, the tissue is a normal tissue. In another example, the tissue is neoplastic (e.g., tumorous or metastatic).

The method can further include storing the substrate for at least 12, 24, 48, 100, or 200 hours prior to the transcribing, e.g., and in some cases at least 6 months, or at least a year.

In one embodiment, the promoter primers are the same. At least some of the promoter primers can include a T7 promoter, a homopolymeric T tract, and a terminal A, G, or C. In one embodiment, the promoter primers are covalently attached to the substrate, e.g., by their 5′ end. In another, they are non-covalently attached.

The RNA replicates can be labeled. The method can further include hybridizing the labeled RNA replicates to a target, e.g., a filter, a nucleic acid array, or a solution comprising target nucleic acids.

The substrate can be a surface of a well of a multi-well plate. The substrate can be at least partially composed of glass or a plastic.

In one embodiment, the method further includes hybridizing a labeled probe to the substrate.

In another aspect, the invention features a method that includes: providing a substrate having promoter primers; annealing a sample that comprises RNAs to the substrate; extending the promoter primers using an RNA-directed DNA polymerase to construct DNA replicates of the RNAs; synthesizing DNA strands complementary to the DNA replicates; ligating an adaptor to the DNA replicates, and transcribing the complementary strand using an RNA polymerase that recognizes the promoter region to produce RNA replicates. The adaptor can include a promoter region for a second RNA polymerase. The adaptor can further include a unique restriction enzyme recognition site, a translational control sequence, or a sequence encoding a purification tag.

The method can further include reverse transcribing the RNA replicates to form second DNA replicates and transcribing the second DNA replicates using the second RNA polymerase.

In still another aspect, the invention features a method that includes: providing a substrate having a promoter primers; annealing a sample that comprises RNAs to the substrate; and extending the promoter primers using an RNA-directed DNA polymerase to construct DNA replicates of the RNAs. In particular, the invention features a substrate made by a method described herein, such as one of the afore-mentioned methods.

The invention also features a kit that includes an array of sense probes and an array of anti-sense probes, wherein for each of at least 10, 20, 30, 40, 60, or 80% of the probes on the array of sense probes, a corresponding and complementary probe is present on the array of anti-sense probes.

In another aspect, the invention features a method that includes: providing a nucleic acid sample; preparing a first and second population of single-stranded nucleic acid strands, wherein the strands of the first population are complementary to the strands of the second population; and evaluating the abundance of a plurality of species in the first population using first probes and the abundance of a plurality of species in the second population using second probes, wherein the first and second probes are substantially complementary. The strands can be RNA or DNA. In one embodiment, the first probes are attached to a first planar array and the second probes are attached to a second planar array. The method can further include determining a score that is a function of the hybridization level of a given sequence to a corresponding first probe and the hybridization level of a complement of the given sequence to a corresponding second probe. For example, the score can be a function of a ratio of the hybridization levels. The method can further include repeating the method for a second sample and comparing the ratio associated with a given sequence between the first and second sample to the ratio associated with a complement of the given sequence between the first and second sample.

The methods described herein can produce a population of relevant single stranded nucleic acids. The nucleic acids can, for example, all have the same strandedness. In many embodiments, the product nucleic acid is RNA, which can be enzymatically distinguished from input DNA. Thus, any remnant input DNA can be specifically removed by digestion. Moreover, the methods are particularly suited for multiplex analysis, and, thus, adaptable for applications such as the high-throughput analysis of multiple nucleic acid polymorphisms. The challenges of multiplex analysis are described, for example, in Pastinen et al. ((2000) Genome Research 10:1031-1042).

Further, as many embodiments of the invention do not require PCR or another thermal cycling reaction, many and sometimes all steps can be conducted under isothermal conditions, typically at temperatures such as 4° C., 16° C., 25° C., 37° C. or 42° C. Reactions can times, e.g., at least 1, 2, 4, 6, or 12 hours.

Still another advantage is that the methods are readily adapted to amplify DNA rather than RNA. In particular, genomic DNA or cDNA can be analyzed, for example, for polymorphisms. cDNA can be obtained from a single cell, or from a small number of cells (e.g., less than 10⁶, 10⁵, or 1000, 100, or 50 cells).

The invention also provides substrates that are effectively “promoter primer chips.” These chips can be produced in quantity and used to query a relevant subset of a genome. Further, as set forth below, once primed with sample nucleic acids or ligated to sample nucleic acids, the chips can be stored, thereby archiving the sample. Later, the stored chips can be used for additional nucleic acid production.

The chips and other substrates also advantageously concentrate relevant target nucleic acids from a complex sample. The removal of non-relevant nucleic acids from a complex sample, before initiating amplification, can further reduce the likelihood of background signals. A background element which appears early in an amplification cycle can dominate species of interest. Some RNA polymerases, such as T7 RNA polymerase, can produce >600 copies of each template in one transcription reaction. Therefore, two to three cycles of transcription-based amplification can achieve very high yields. As exemplified below, the method is highly sensitive. For example, a specific nucleic acid fragment can be amplified from 100 ng of human genomic DNA or from a cDNA, e.g., from a single cell.

The method enables, among other things, the production and archiving of a reproducible nucleic acid library without the use of cells. The library can be stored, e.g., as an immobilized population of nucleic acids. Because the nucleic acids are not introduced into cells, representation of nucleic acids in the library is not subjected to biases that can be caused by cellular toxicity and other unpredictable factors.

In addition, as described for some methods, use of a substrate (such as a pin or an array) enables simple exchange of reaction solutions. For example, enzymes can be removed without complex steps such as heat inactivation, phenol extraction, or ethanol precipitation.

With respect to many embodiments, it is also found that transcription-based amplification can include designing the promoter position to create a defined terminus for each RNA product. Probes for each product are similarly designed.

An “oligonucleotide” refers to a nucleic acid of less than 150 nucleotides. Oligonucleotides can be produced synthetically or enzymatically (e.g., by excision from a larger nucleic acid). An oligonucleotide can include a double-stranded region, e.g., by self-annealing or by annealing to another nucleic acid. An oligonucleotide is typically a DNA molecule, e.g., with an extendable 3′ end. An oligonucleotide can include one or more modification (e.g., attached ligands).

A “primer” is a nucleic acid that can be extended by a polymerase.

A “capture probe” is a probe that includes a nucleic acid that can interrogate another nucleic acid and that can alter the relationship between that other nucleic acid and substrate. For example, the capture probe can retain that other nucleic acid on the substrate. Capture probes can be physically attached to the substrate by a covalent or non-covalent bond.

The details of a number of embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic of an exemplary substrate 100. The substrate 100 includes a promoter primer 110 that includes a promoter sequence 112 and a sample nucleic acid binding sequence 114 and a capture probe 120 that includes a target binding sequence 122.

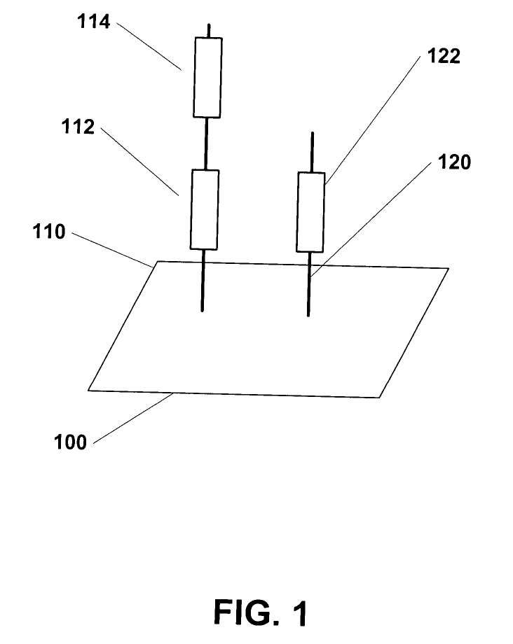

FIG. 2 is a schematic of an exemplary detection method.

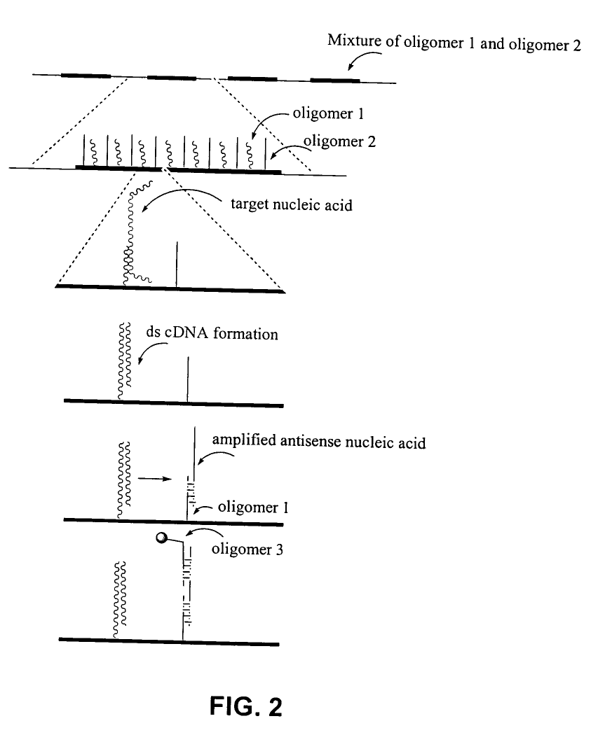

### DETAILED DESCRIPTION

In one aspect, the invention provides a substrate that can function to both amplify nucleic acid and detect amplification products. In one embodiment, the substrate includes a promoter primer and a capture probe. The promoter primer can be used to form a template nucleic acid, e.g., as described herein. The capture probe can be used to detect nucleic acids transcribed from the template nucleic acid or derivates thereof.

Transcription-Based Amplification

For an embodiment in which mRNA is evaluated, for example, the promoter primer includes target annealing sequence having poly-dT. In some embodiments, the target annealing sequence includes a single A, G, or C nucleotide at its 3′ terminus. The A, G, or C nucleotide serves to anchor the poly-dT primer at the 5′ end of the poly-A tract of mRNA. Typically, the substrate includes a population of immobilized oligonucleotides with different terminal nucleotides, e.g., so that all mRNAs can hybridize to the target annealing sequence. Dinucleotide anchors can also be used, as can gene or family specific primers. Other target annealing sequences can also be used (e.g., target specific nucleic acid sequences or repetitive sequences, e.g., as for some genome nucleic acid sequences).

The substrate can be washed and equilibrated in 1× first strand synthesis buffer (e.g., 1× first strand synthesis buffer (50 mM Tris-HCL, pH 8.3 at 42° C.; 50 mM KCl; 10 mM MgCl₂; 0.5 mM spermidine; 10 mM DTT). A sample is contacted to the substrate so that nucleic acids in the sample can anneal to the promoter primer. For example, here the poly-A tract of mRNA would anneal to the poly-dT target annealing sequence. The annealing can proceed at 42° C. for at least 5 minutes.

After annealing, cDNA synthesis is initiated by the addition of sodium pyrophosphate, AMV reverse transcriptase (e.g., from UNIVERSAL RIBOCLONE™ cDNA Synthesis System Catalog No. C4360 from Promega Corp, Madison, Wis., USA) and deoxynucleotides (e.g., 1 mM each of dATP, dCTP, dGTP, dTTP). The reaction can proceed, e.g., at 42° C. for at least 30 minutes.

After synthesis of the first cDNA strand, a variety of methods can be used to produce the second cDNA strand, if required. See below (“Second Stand Synthesis”). The substrate now has attached cDNA copies of each annealed mRNA. The cDNA copies can function as immobilized templates since they are operably linked to a promoter. If desired, the substrate can be stored at this stage, and then retrieved for later amplification and analysis. Typically, the substrate is washed extensively and incubated in a cryoprotectant (e.g., 10% glycerol) or in buffer (e.g., with preservatives, such as sodium azide), prior to storage.

The substrate can be used to generate RNA replicates from the template. The support is first equilibrated in RNA polymerase transcription buffer and then contacted with RNA polymerase transcription reagents, e.g., T7 RNA polymerase and ribonucleotides (e.g., as provided by AMPLISCRIBE™ T7 High Yield Transcription Kit, Catalog No. AS2607, Epicentre, Madison, Wis.). Reactions are appropriately incubated, e.g., at 37° C. for at least 5 minutes. The RNA replicates can be labeled, e.g., by including at least one labeled ribonucleotide.

The amplification product (e.g., RNA or a derivative thereof) can interact with the capture probe and the interaction can be detected, e.g., as described herein.

Capture Probes

For embodiments that detect transcripts, a capture probe can be designed so that it includes a segment that specifically interacts with the sense strand of a particular target nucleic acid. To detect other nucleic acids (e.g., genomic DNA or viral DNA), a capture probe can be designed so that it includes a segment that specifically interacts with the amplified strand. For example, the segment can be exactly complementary or it can be sufficiently complementary to have a Tₘ of greater than 56, 60, 62, 64, 68, or 70° C. and/or less than 75, 70, 68, or 64° C. The segment can be at least 9, 10, 15, 20, 25, 30, 40, 100, or 1000 nucleotides in length, and ranges therebetween.

In one embodiment, the capture probe does not include a sequence that interacts with the promoter primer. However, in another embodiment, the capture probe and the promoter primer are both immobilized in a configuration that prevents undesirable interactions between the capture probe and the promoter primer.

In one embodiment, the capture probe is covalently attached to the substrate. The capture probe may also be non-extendable. For example, it may not have a 3′ OH group or it may be attached by its 3′ O to the substrate or a blocking group. A non-extendable probe may eliminate the possibility that the capture probe is extended during promoter primer extension.

The molar ratio of the promoter primer to the capture probe can be 1:1, 1:10, 1:100, 1:1000, and 1:10,000. The appropriate ratio can be chosen depending on the desired sensitivity and saturation levels. The promoter primer and the capture oligomer can be located on in the same region of the substrate or at separate subregions of a region of the substrate.

The capture probes can be located in a variety of positions relative to the promoter primer. In one embodiment, the capture probes and the promoter primers are intermingled. For example, a solution is prepared includes both the capture probe and the promoter primer. The solution is spotted on the substrate such that both the capture probe and the promoter primer are immobilized. In another embodiment, the capture probe and the promoter primers are positioned in separate subregions, but within a certain distance of one another. For example, for planar substrates, the capture probe may be positioned in a spot adjacent to the promoter primer. In another example, the capture probe may be positioned in an annulus surrounding the promoter primer. In still another example, different capture probes are positioned around the promoter primer. They can be located, e.g., at the points of a square, triangle, pentagon, hexagon, or octagon surrounding the promoter primer or checkered around one or more promoter primers. In another example, the promoter primer and the capture probe are located at separate subregions that are in fluid communication, e.g., by means of a channel, e.g., a microchannel such as a groove etched into the substrate.

In an embodiment in which a non-planar substrate is used, the capture probes may be located on one surface and the promoter primers are located on another surface. For example, for a well of a microtitre plate, the promoter primers can be located on the walls of the well and the capture probe can be located on the bottom surface. This configuration can aid detection and imaging since interaction between a detection probe and the template nucleic acids would occur on the walls of the well and would not obscure the bottom surface.

Detection Methods

A variety of methods can be used to detect interaction between amplified nucleic acid and a capture probe.

In one embodiment, the amplified nucleic acids are labeled, e.g., with a chemically altered nucleotide (e.g., fluorophore, digoxygenin or biotin labeled) or a radiolabel (e.g., ³⁵S, ³³P, ³²P, ¹⁴C). Labeled nucleotides are only added to the primer in an extension reaction, e.g., using reverse transcriptase, if the primer is complementary at the query site. After amplified nucleic acids are produced, the substrate can be washed to remove amplified nucleic acids that do not bind to a capture probe under the wash conditions (e.g., using a selected degree of stringency). After washing, amplified nucleic acids that anneal to the capture probe can be detected by detecting the label. For example, the substrate can be imaged, e.g., exposed to photographic film, evaluated using a camera, or scanned. Digoxygenin and biotin labeled nucleotides can be detected, e.g., using reagents that specifically bind to such chemical tags.

In another embodiment, a detector probe is used in addition to a capture probe. The amplified nucleic acids are typically unlabeled (but may also be labeled, e.g., so they can be detected in a different signal channel). The amplified nucleic acids are hybridized to a detector probe that includes at least one region that hybridizes to an intended target in a segment other than the region that is bound by the capture probe. The order of addition of the detector probe can vary. For example, the detector probe may be present on the substrate prior, during, and/or after the transcription that produces the amplified nucleic acids. Typically the detector probe is labeled so retention of the detector probe on the substrate indicates presence of a target nucleic acid. This assay provides for two specificity steps:—(1) interaction between the capture probe and the target nucleic acid and (2) interaction between the detector nucleic acid and the target nucleic acid.

In one embodiment, the substrate includes a plurality of different capture probes at distinguishable positions. To analyze a profile of sample nucleic acids, labeled RNA products can be generated from a template array to replicates of the sample nucleic acids. The replicates are hybridized to a detection array that includes a plurality of capture probes. The detection array can be imaged (e.g. scanned) to determine whether and to what extent the labeled RNA products hybridize to the probes. Because each probe is at a unique address, the amount of each species can be inferred. Methods for hybridization to detection arrays are well known. The information obtained by analyzing the detection array can be stored in a machine-accessible medium, e.g., with a pointer to information about the location or identify of an archival template array that can be used to make RNA replicates of the sample nucleic acid. If the detector nucleic acid includes a label, its presence on the array can be detected using methods appropriate for that label.

In one implementation, the detector nucleic acid and the capture probe each include a label. The labels on the respective nucleic acids can interact. For example, the labels can be designed for proximity detection so that, when the label on the detector nucleic acid and the label on the capture probe are in proximity, a signal or a change in signal can be detected. Proximity of the label can be detected by diffusion of an agent (e.g., singlet oxygen, see, e.g., Beaudet et al. (2001) Genome Res. 11(4):600-8) or by fluorescence resonance energy transfer or quenching.

Other methods for detecting a nucleic acid bound to a capture probe include mass spectroscopy, e.g., of the bound target nucleic acid or of a detector nucleic acid whose mass signature may be known, and surface plasmon resonance.

Spacer Lengths

In one embodiment, template nucleic acids are prepared such that the promoter sequence is spaced from the substrate. For example, the distance between the 5′ terminus of the promoter and the substrate is at least 4 nm. The distance can be achieved by appropriately spacing the promoter primer from the substrate.

1) Non-Covalent Immobilization:

Promoter primers can be immobilized by non-covalent interaction between a ligand that is covalently attached to the oligonucleotide and a protein immobilized on the support. For example, the ligand can be biotin and the interaction can be between biotin and a biotin-interacting protein (e.g., streptavidin or avidin). When this configuration (ligand/ligand-binding protein, e.g., biotin/biotin-interacting protein) is used to immobilize the oligonucleotide to the substrate, optimal spacings between the immobilized nucleotide position (e.g., the 5′ nucleotide) and the 5′ end of the promoter sequence is at least 5 nucleotides, e.g., between 6 and 20 nucleotides, or between 6 and 15, or 6 and 12 nucleotides, e.g., 6, 7, 8, 9, 10, 11, or 12 nucleotides. In one embodiment, it is also possible to substitute the nucleic acid spacer sequence with a non-nucleic acid spacer, e.g., a chemical linker described below.

Other examples ligand/ligand-binding protein interactions include: FK506 and FK506BP, chitin and chitin binding protein; cellulose and cellulase (CBD); amylose or maltose and maltose binding protein; methotrexate and dihydrofolate reductases.

2) Covalent Immobilization

Promoter primers can be immobilized by covalent coupling chemistries, e.g., by a homopolymeric linker (e.g., a polyethylene glycol linker), and phosphate linkages. Additional examples of homopolymeric linkers include: polymers with subunits having 2, 3, or 4 main chain atoms, and between 5 and 12 repeats of the subunits. For example, the linker can be composed of polyethylene glycol ((CH₂CH₂O)ₙ—, e.g., where n is >8, e.g., 8 to 20, 8 to 16 or 8 to 12); ((CH₂)ₘO)ₙ—, e.g., where n is >8, e.g., 8 to 20, 8 to 16 or 8 to 12) and where m is >1, 2, 3 and <12, 8, 6, or 5; and/or polymethylene, ((CH₂)ₙ—, e.g., where n is >18, e.g., 18 to 60, or 24 to 48). Generally it is possible to use any chemical linker (e.g., without nucleotide units) that has the same physical length or atom length as a linker described herein, e.g., a homopolymeric linker described above, e.g., including 18 to 60, or 24 to 48 main chain atoms.

An oligonucleotide that includes a nucleotide that is directly coupled to the immobilized support can include a spacer of between 15 and 45 nucleotides, e.g., between 20 and 35 nucleotides, e.g., between 23 and 28 nucleotides, or about 25 nucleotides. The oligonucleotide can be coupled, e.g., using carbodiimide activation.

In one embodiment that uses carbodiimide activation, a primer oligonucleotide containing a 5′-phosphate group is activated with ethyl 1,1-dimethylaminopropylcarbodiimide hydrochloride (EDC) in the presence of 1-methylimidazole. See, e.g., Chu et al. (Nucleic Res., 11, 6513 (1983). The active phosphate ester intermediate is converted into a reactive phosphorimidazolide which reacts spontaneously with an amino group on the substrate. This covalent coupling chemistry further ensures that only the 5′-end of the primer oligonucleotide is attached to the substrate.

Other attachment chemistries include: coupling between an amine-including oligonucleotide and an activated carboxylate group or succinimidyl ester; coupling between a thiol-including oligonucleotide (SH-oligo) and an alkylating reagent such as an iodoacetamide or maleimide; coupling of an Acrydite-oligonucleotide through a thioether. See, e.g., Adessi et al. (2000) Nucleic Acids Research 28:e87; Ghosh, and Musso (1987) Nucleic Acids Res. 15:5353-5372; Lindroos et al. (2001) Nucleic Acids Res. 29:e69; Rogers et al. (1999) Anal. Biochem. 266:23-30.

Nucleotide regions of spacers can be prepared using any nucleic acid sequence, for example, a homopolymeric sequence, a low complexity sequence, a medium complexity sequence, a complex sequence, or a sequence absent from the sample of relevance. A “low complexity sequence” is a sequence that includes repeating units of 4, 3, or 2 nucleotides. A “medium complexity sequence” includes fewer than three types of repeats, each repeat being 2, 3, 4, or 5 nucleotides. A sequence absent from a sample of relevance can be identified by searching a computer database of sequences potentially in the sample. For example, if the sample is human, it is possible to identify a spacer that is not present in human genome sequence or that is not complementary to any human genome sequence.

Capture probes can be similarly spaced from the substrate.

Second Strand Synthesis

One method includes reaction promoter primer-cDNA nucleic acid hybrids with a strand-displacing DNA polymerase (e.g., DNA polymerase I or phi29 DNA polymerase) or Klenow and RNase H, e.g., at 16° C. RNase H is used to nick the RNA strand. If necessary, the reaction can be completed by T4 DNA polymerase. The second cDNA strand forms homoduplexes of DNA on the array and thereby contributes to stability.

Another method includes tailing of the extended immobilized oligonucleotide by terminal transferase, e.g., in the presence of dGTP so that a polyG tail is add, e.g., about 10-20, 12-18, or an average of 15 nucleotides in length. After the tailing, a primer that includes C is annealed, and optionally ligated, and extended. See generally, e.g., Okayama and Berg Mol. Cell. Bio. 2:161-170, 1982; Spickofsky and Margolskee Nucleic Acids Res. 19: 7105-7111, 1991); Dugaiczyk, et. al. Biochemistry 19:5869-5873, 1980).

It is also possible for a hairpin to form spontaneously such that the terminal nucleotide can be extended to form the complementary strand.

Still another method uses primer corresponding to the 5′ end of the target sequence, e.g., complementary to the 3′ end of the extended oligonucleotide. Random hexanucleotides or other priming sequences can also be used, particularly after removal of the RNA strand, e.g., by denaturation or nicking with RNase H.

Promoter Primer Design

Promoter primers are used to attach a promoter for an RNA polymerase to another nucleic acid sequence to prepare a DNA template. In one embodiment, the promoter primers used in this method generally have a length of 25 to 100 nucleotides, e.g., about 30 to 50 or 40 to 60 nucleotides. The promoter primers are also designed so that the promoter sequence is spaced from the substrate when the promoter primer is immobilized. The spacing can be as described above.

An promoter primer has a 3′ sequence, also termed “target binding region,” which anneals to a target site within a target fragment. This sequence can be substantially homologous, e.g., 90 to 100% identical, to the target site. An identical or nearly identical sequence increases specificity of amplification. The length of the target binding region can be selected such that the Tₘ for a duplex formed between it and the target site is at least about 42° C., 50° C., or 55° C. The target binding region can be optimized such that it does not anneal to itself or the remainder of the promoter primer (e.g., form hairpins).

An promoter primer also contains a promoter sequence 5′ end to the target sequence. The promoter sequence is recognized by an RNA polymerase. The RNA polymerase can be prokaryotic, eukaryotic, or archeal. For example, the RNA polymerase can be a prokaryotic bacteriophage RNA polymerase such as the T7, T3, and SP6 RNA polymerases. Hence, exemplary promoter sequences include, but not limited to, T7, T3, Sp6 RNA polymerase promoters sequences. Generally, any RNA polymerase that can be specifically directed to a promoter can be used. For example, SP01 promoters can be used in conjunction with sigma factors from the Bacillus subtilis phage SP01 to target RNA polymerase to SP01 promoters.

The promoter primer can be attached or attachable to a substrate. For example, the promoter primer can also include a modification to facilitate affinity capture of the target or of the duplex formed by extension of the target-promoter primer complex. The modification can include, for example, a small molecule of less than 1000 Daltons molecular weight for binding by a protein that binds (e.g., specifically binds) to the small molecule. For example, one or more biotinylated deoxynucleotides (or other ligands) can be used. Other useful modifications include amino and thiol moieties. A biotinylated moiety can be bound to immobilized streptavidin or avidin. Other useful non-covalent and covalent linkages are widely known. For some reactions, e.g., using a biotinylated promoter primer, about 0. 1, 1, 5, 10, 20, or 100 pmol of promoter primer are used per reaction. The reaction might be about the size of a well of a 96-well carrier.

In one embodiment, the primer includes a sequence have one strand of a restriction enzyme recognition site. The primer can also include a modified base, such as α S-dNTP, within the recognition site, e.g., such that the primer strand is not cleaved. DNA polymerase will then recognize the nick, and start polymerization which results in displacement of the nicked DNA strand. Repeat nicking and polymerization lead to linear amplification of one strand of the target DNA.

Optionally, a linker sequence can be included between the promoter primer promoter sequence and the target sequence. The linker sequence is transcribed, and can include restriction endonuclease sites (e.g., sites for a 6- or 8-base pair cutter) to facilitate cloning of the amplified nucleic acids, a synthetic identification tag, or a universal sequence. The linker region can include a sequence that is recognized by an RNA binding protein when the linker region is transcribed into RNA. Exemplary RNA binding proteins include Tat and Nus.

In one embodiment, the linker region includes an internal ribosome entry site, an initiator methionine, an epitope tag, a purification or detection tag, and/or a translational regulatory sequence. Promoter primers can be synthesized using standard oligonucleotide synthesis chemistry, either in situ on a substrate or prior to attachment to a substrate. In another implementation, promoter primers can be produced enzymatically (e.g., by PCR) or by isolation from a host cell (e.g., E. coli).

Promoter Primer Annealing

The promoter primers are annealed to a sample nucleic acid which functions as a template for extension. The annealing can be performed at a temperature below the Tₘ of the promoter primer for its target binding site. Hybridization of promoter primers to the single stranded target fragments can be performed in any container, e.g., a tube, such as a micro-centrifuge tube, a well, or a flow cell. The promoter primer can be attached to a substrate, either before, during, or after annealing. A variety of hybridization conditions can be used. Hybridization conditions are described, for example, in standard laboratory manuals such as (Molecular Cloning, 3^{rd} edition, Cold Spring Harbor Press, ed. Sambrook & Russell). Temperature and salt concentration can be selected to achieve the desired stringency.

One method is to hybridize the single-stranded targets to 5′→3′ directionally anchored promoter primers. After hybridization, unbound DNA can be removed by washing with buffers.

Template Extension

DNA polymerase is used to append the promoter primer to the target sequence by primer extension, thereby forming double-stranded DNA. Exemplary DNA polymerases include the Klenow fragment (3′-5′ exo⁻), DNA polymerase I, and SEQUENASE™ 2.0 (Amersham Pharmacia Biotech). Any DNA polymerase may suffice, particularly those lacking 3′ to 5′ exonuclease activity. Conditions for double-stranded DNA synthesis are described, for example, in Gubler (1987) Methods Enzymol 152: 330-335.

The DNA polymerase can extend the annealed target nucleic acid segment using the promoter (or other non-target binding region) of the promoter primer as a template. This step renders the promoter region double-stranded and functional. Further the extension process “operably links” the promoter to the target fragment. As used herein, the term “operably linked” refers to a functional linkage between the affecting sequence (typically a promoter) and the controlled sequence.

Typically, the promoter primer is extended so as to render both the promoter and the target double stranded. The extended nucleic acids can be stored as DNA duplexes. Such stored nucleic acid has the advantage of conformational and chemical stability.

Since a double stranded region is optional in the region after the +1 site of the promoter, at least for the bacteriophage RNA polymerases such as SP6, T7, and T3, in some embodiments, a promoter primer is substituted with a promoter oligonucleotide that includes an unextendable the 3′ end. In some of these implementations, only the promoter region is rendered double-stranded. The promoter oligonucleotide is not used as a primer, but as a template for forming a double-stranded promoter region. In related implementations, an immobilized double-stranded promoter segment used and a target nucleic acid is ligated to the promoter segment to provide a transcribable template nucleic acid.

Ligation

In another embodiment, the target fragment is ligated to the bottom strand of an SSP duplex, which includes both the promoter primer, and a complementary strand. The three component strands can be added in any order. Since the template for transcription can be single stranded (see below), so long as the promoter is double-stranded, the asymmetric hybrid formed by the three component strands is sufficient for transcriptional amplification. Two components can also be used, for example, if the SSP duplex is formed from a hairpin nucleic acid that includes the promoter primer sequences and the complementary region.

Amplification by Transcription

The T7 polymerase polypeptide can be produced from the cloned gene, the T7 gene 1, see e.g., U.S. Pat. No. 5,869,320 (Studier et al.). T7 RNA polymerase can be purified from induced cells that have a nucleic acid for T7 gene operably linked to an inducible promoter. Chamberlin et al., (1970) Nature, 228, 227-231 describes one exemplary scheme for purifying the polymerase.

T7 RNA polymerase is highly specificity for its promoter site (Chamberlin et al., in The Enzymes, ed. P. Boyer (Academic Press, New York) pp. 87-108 (1982)). The T7 polymerase recognizes a highly conserved sequence spanning about bp-17 to about +6 relative to the start of the RNA chain (Dunn and Studier, (1983) J. Mol. Biol. 166: 477-535 and (1984) J. Mol. Biol. 175: 111-112. In one embodiment, the promoter includes the −17 to +1 region. Typically, the promoter region of the template strand is double-stranded DNA. The remainder of the template may be either single-stranded, double-stranded, or combinations thereof.

T7 RNA polymerase is particularly useful for amplification of diverse nucleic acid sequences as a result of the dearth of efficient termination signals for T7 RNA polymerase (see, Rosenberg et al., (1987) Gene 56: 125-135. The T7 RNA polymerase is available, e.g., from Promega Biotech, (Madison, Wis.) and Epicentre Technologies, (Madison, Wis.).

SP6 and T3 RNA polymerases have similar properties. Further, each of these three polymerases is highly specific as it does not transcribe non-cognate promoters. The minimal efficient promoter sequences for these polymerases are listed in Table 1 below. The +1 nucleotide is underscored. Other prokaryotic promoters can be used, e.g., a promoter recognized by an E. coli RNA polymerase. The 5′ end of a promoter can be defined, e.g., by mutational analysis (e.g., deletion mapping), wherein the 5′ end bounds the minimal region that is sufficient to provide at least 70% of the wildtype promoter's activity.

TABLE 1
Bacteriophage RNA Polymerase Promoters

| | |
|---|---|
| RNA polymerase | Specific Promoter Sequence |

| | | |
|---|---|---|
| T7 | TAATACGACTCACTATAGG | (SEQ ID NO:23) |
| T3 | AATTAACCCTCACTAAAGG | (SEQ ID NO:24) |
| SP6 | ATTTAGGTGACACTATAGA | (SEQ ID NO:25) |

To obtain amplified RNA, RNA polymerase reaction buffer, excess of all four ribonucleotides, and the corresponding RNA polymerase enzyme are added, and incubate at 37° C. for 25 mins-24 hours. In vitro transcription is described, e.g., in Melton, D. et al. (1984) Nucl. Acid. Res. 12:7035. The transcription reaction buffer can include a variety of components, e.g., including:
1 to 20 mM NaCl
1-40 mM MgCl₂ (e.g., 24, 34, or 40 mM)
10 to 50 mM Tris·HCl (pH about 7.3, 7.4, or 7.5)
1, 2, 3, 5, 7.5, 10 mM rNTP
1, 3, 5, 10 mM DTT
2 U/μl SUPERASE-IN™ inhibitor

To obtain labeled RNA to be used as hybridization probe in sequence analysis, one or more labeled ribonucleotides are also added. Depending on the intended detection method, the labels can be, but not limit to, fluorescent dyes such as fluorescein and the cyanine dyes (Cy3, Cy5, Alexa 542, and Bodipy 630/650); radiolabels such as ³²P, ³³P, ³⁵S, and ³H; colorimetric or chemiluminescence; and binding pair components such as biotin or digoxygenin.

Post Processing, Archiving, and Storage

The method can further include any of a number of post-processing steps. For example, the RNA products can be reverse transcribed into DNA using specific or random primers. Clearly, the RNA products can be used for a variety of purposes. For example, the RNA products (if they have the appropriate strandedness) can be translated, and the translation products analyzed, e.g., for an activity or by contacting the translation products with an antibody. Translation products can be analyzed, e.g., to evaluate one, two, three or more criteria about each product, e.g., using gel electrophoresis, 2D gel electrophoresis, mass spectrometry, and other methods. The information can be stored in a database, e.g., using a record that includes two, three or more fields, e.g., to provide a multidimensional vector.

The RNA can be quantitated, e.g., to determine the abundance of different species. If the RNA is labeled, it can be hybridized to an array of positional probes for the different known RNA species. In some cases, the RNA is itself functional, e.g., the RNA is an aptamer or a catalyst. Such RNA can be analyzed for binding or catalytic properties.

Anchored, promoter appended DNA target can also be reused and/or stored for future reference. For example, if a chip of promoter primers is used, the chip can washed free of reagents. The washed chip can either be immediately reused for additional rounds of transcriptional amplification or stored, e.g., in an archival process. A stored chip can be dehydrated and frozen, or coated with a cryoprotectant such as a glycerol solution, and frozen, or store in buffer+preservatives such as sodium azide at 4° C. When desired, a stored chip can be retrieved, washed, and applied with fresh reagents for transcription, e.g., ribonucleotides and the appropriate RNA polymerase. As described below, a variety of substrates (e.g., pins, microtitre wells, spin cups, matrices, and membranes) can be used.

Likewise, with respect to the cyclic TCR method, in which two different sets of templates (e.g., T7 and T3) are produced, the templates may be archived separately or together.

By coupling templates from different cycles to separate supports, a master and working set of templates can be generated. The working sets can be distributed to different users (e.g., customers). The master set can be used to produce additional working sets and may also be stored for reference or quality control.

Adaptor Addition

After second cDNA strand synthesis, in some embodiments, a DNA adaptor is ligated to the free terminus of the immobilized cDNA. The DNA adaptor can include a transcription promoter, e.g., the T3 DNA polymerase promoter. This design is useful for a transcription chain reaction or for the addition of a tag or regulatory sequence to the RNA. The adaptor can also include one or more of a restriction site (e.g., a 4-, 6- or an 8-base cutter such as AscI), a sequence encoding a purification tag (such as the hexa-His tag or S-tag), a splicing sequence, and a translational control signal (such as the Kozak consensus sequence or other ribosome entry site) or complements thereof.

Transcription Chain Reaction (TCR)

Increased amplification is achieved by using the RNA products as templates for additional transcription. The RNA products are converted to DNA by reverse transcription in a format analogous to the process described above for promoter primer directed synthesis. The RNA transcripts made from the promoter primer appended double stranded DNA are captured by a second promoter primer which contains a sequence complementary to the newly synthesized RNA strand, e.g., at the 3′ end of the RNA strand. The promoter segment of the second promoter primer can be different from the promoter of the first promoter primer. The captured RNA can now be converted to double-stranded DNA by reverse transcriptase and DNA polymerase.

Transcription from these newly synthesized DNA produces RNA, and results in enhanced amplification. The method can be used to detect sequences at very low concentrations, e.g., from a single cancer cell in a population of normal cells.

As described above, as the nucleic acid promoter-target fusions are captured, the substrate attached to the first and second promoter primers can be stored for later rounds of transcription.

In one embodiment, the substrate contains multiple pairs of first and second promoter primers, e.g., to amplify multiple different targets.

RNA products from an initial amplification stage are reverse transcribed, e.g., using a target specific primer. The DNA strand from reverse transcription can be rendered single stranded, e.g., by mild alkali hydrolysis, heat treatment, 50% formamide at 50° C., or ribonuclease digestion, e.g., using RNaseH. The single stranded DNA replicates can then anneal to the available immobilized promoter primers. The process allows for enhanced amplification.

A fragment containing a single nucleotide polymorphism at a query site is amplified using the SSAT method. Then, RNA products are hybridized to immobilized reverse transcription primers. The reverse transcription primers position their ultimate 3′ nucleotide opposite the query site. Reverse transcription only proceeds if the ultimate 3′ nucleotide is complementary to the query site nucleotide. The incorporation of label, e.g., a Cy3 labeled dNTP can be used to monitor the process.

Cycles of T7 (left) and T3 (right) amplification are shown. mRNA or sRNA (from a cycle of T3 amplification) are hybridized to promoter primers (e.g., T7-d(T)nV) that are attached to support. The promoter primer can include a spacer sequence between the promoter and the substrate attachment site. For example, the spacer can be at least 6, 12, 18, or 24 nucleotides in length. Template DNA is produced, e.g., by cDNA synthesis. An adaptor molecule is ligated to the template in an initial cycle (e.g., this is optional if sRNA is used). The adaptor preferably includes a tag sequence that is absent from the sample. A computer program can be used to predict sequences that should be absent from a sample obtained from a particular organism, e.g., by comparison to a comprehensive database of genomic or cDNA sequences from that organism. After ligation of the adaptor, transcripts (aRNA) are produced using T7 polymerase.

The transcripts include the tag sequence as well as the sequence from the sample nucleic acid. Transcripts are then hybridized to a T3-TCR promoter primer attached to the same support or another substrate. Again, cDNA is produced from the annealed transcripts. The T3 polymerase is now used to produce sRNA. The sRNA can be cyclic deployed to produce additional aRNA transcripts.

Joining of the adaptor can be implemented in a variety of ways. For example, DNA and RNA ligases can be used (e.g., to ligate a preformed duplex that includes the tag sequence). In one embodiment, terminal transferase and dGTP are used to add a homopolymeric G tail to the DNA strand. An oligonucleotide that includes the tag sequence and a 3′ polymeric C tail is annealed and used to prime synthesis of the second DNA strand.

In some implementations, it is also possible to detect a nucleic acid replicate made from a template by hybridization of a probe that is complementary to an adaptor sequence.

Substrates

As described herein, many embodiments include a substrate that includes a promoter primer and a capture probe. For example, the substrate can be composed of any insoluble material. In one example, the substrate is a rigid planar device such as a chip (e.g., a glass slide, e.g., a microscope slide). In another example, the substrate is a reaction vessel such as a multi-container sample carrier (e.g., a microtitre plate), tube, column, spin-cup, disposable pipet tip, ring, disc (e.g., paper disc), lantern, pestle, membrane, or portions thereof For example, the templates can be attached to a surface within one or more microtitre wells (e.g., in a variety of formats, including single, strips, 96-well, 384-well, robotically manipulated single or multiple plates). The microtitre plates can conveniently be placed into thermocontroller units, e.g., thermocyclers in order to finely control reaction temperatures.

In one embodiment, a spin cup is used. The cup has a porous membrane, e.g., a 0.45 μm membrane or any size membrane that facilitates passage of macromolecules such as the reaction enzymes. To conduct a set of multiple reactions, the reaction components are passed through the membrane (e.g., by low-speed centrifugation). To switch reaction components, buffer or a subsequent reaction mixture is washed through the membrane. The promoter primer is first physically attached to the membrane (e.g., by a non-covalent or covalent linkage). Thus, throughout the reactions the promoter primers and templates that incorporate them remain within the membrane of the spin cup. After templates are generated, the membranes can also be archived, e.g., for subsequent RNA transcription. Transcription products can also be collected by low speed centrifugation of the spin cup. If the spin cup further includes a capture probe, such centrifugation can be used to separate nucleic acids that interact with the capture probe from those that do not.

In another embodiment, a pin or set of pins is used. The promoter primers are physically attached to the pins. The pins can also include a capture probe. For example, the promoter primers are biotinylated and the pin surface is coated with a streptavidin. To process multiple reactions, multiple pins can be rigidly fixed to a holding unit. The holding unit is used to transfer to pins to different reaction mixtures. For example, the holding unit can be a lid of a microtitre plate. The lid is placed on different plates, each plate including appropriate reaction mixtures (e.g., for sample hybridization, reverse transcription, second strand synthesis, and transcription). For cyclic TCR, alternating pins, each pin for capturing T7 and T3 templates.

Substrates can be evaluated by hybridizing a probe that contains a sequence complementary to the primers on the support. For example, to detect the target binding activity of an immobilized primer, a probe oligonucleotide of 5′-biotin-GCGCCAATTATCGAAAAAAAAAAAAAAAAAAAA (SEQ ID NO:27) is allowed to hybridize with the promoter primer on the substrate. The hybridized complex is washed with buffer, and then treated with a streptavidin-horse radish peroxidase conjugate. After the reaction between streptavidin and biotin molecules are completed on the substrate, the unbound enzyme conjugates are washed away. A solution containing o-phenylenediamine and hydrogen peroxide is then added to the substrate. Color development of the solution is measured to indicate the quantity of streptavidin enzyme conjugate bound to the substrate. This in turn indicates the amount of biotin bound to the substrate which indicates the binding activity of the promoter primer on the substrate. Capture probes on the support can be similarly tested, e.g., to test probe specificity.

Fragment Preparation

Another exemplary implementation is directed to the analysis of genomic DNA, e.g., for polymorphisms. The implementation includes: fragment preparation, rendering the sample single-stranded, promoter primer annealing, attachment, transcription, and detection or analysis.

Genomic DNA is isolated from cells, e.g., from a subject such as a human patient. The DNA is digested using restriction enzymes to generate target fragments. To amplify multiple fragments from the genomic DNA, restriction enzymes are selected based on one or more of the following criteria. The target fragments are less than about 2000, 1000, 500, 700, 500, 300, 200 or 100 nucleotides in length. The target fragment includes at least about 15, 18, 20, or 22 nucleotides of non-polymorphic nucleotide sequence in proximity to the restriction site. Such non-polymorphic regions can function as annealing sites for the promoter primer. The polymorphism of interest is located within the central two-thirds of the target fragment. If multiple restriction enzymes are required, the restriction enzymes can be chosen that are compatible, e.g. functional at the same reaction conditions.

For example, enzyme E cuts the DNA into fragments, labeled “a”, “b”, “c”, and “d”. Fragment “d” contains a sequence of interest that, for example, includes a polymorphism represented by a closed circle.

In one embodiment, prior to annealing of the promoter primer to the sample nucleic acids, the cleaved sample nucleic acids are rendered as single stranded. The production of single-stranded DNA can be achieved by heat or chemical denaturation. However, enzymatic means for producing single-stranded DNA were found to be particularly effective for the SSAT method.

The double-stranded DNA fragments are treated with an exonuclease, such as T7 exonuclease or lambda exonuclease. For example, the cleaved sample nucleic acids can be treated with lambda exonuclease for about 1 hour at 37° C.

These exonucleases catalyze digestion of DNA in the 5′ to 3′ direction, thereby sequentially removing 5′ mononucleotides from duplex DNA (Little, J W (1981) Gene Amplification and Analysis 2:135-145; Shimozaki and Okazaki. (1978) Nucl. Acids. Res. 5:4245-4261). The reaction can be inactivated by heating at 75° C. for 30 minutes.

Lambda exonuclease is a highly processive enzyme. As such, it has a strong predilection to remain attached to a substrate DNA strand and digest it to completion before dissociating and attacking another substrate DNA. This feature results in longer single stranded DNA products rather than multiple fragments that are a fraction of the size of the input DNA. The processivity of various exonucleases is described, e.g., in Thomas and Olivera (1978) J Biol Chem 253:424-9.

The processed single stranded DNA products are used as samples for amplification, e.g., as described above.

Software

Also provided is a system and software which can assist, control, and manage one or more steps of a method described herein.

For example, software can include modules for one or more of the following: (1) selecting target nucleic acids (e.g., mRNAs or polymorphisms for analysis; (2) designing promoter primers and/or capture probes for the selected target nucleic acids; (4) interfacing with an oligonucleotide synthesizer or oligonucleotide array synthesizer to produce promoter primers; (5) synthesizing a substrate and (6) receiving and analyzing results from detection of the substrate.

The software can be implemented by a processor running on a networked server or locally on a desktop computer. The processor is interfaced with databases. These databases can be stored in local memory, on machine-readable media, or on remote servers. The processor is also, directly or indirectly, interfaced with external apparati, for example, an array synthesizer, an oligonucleotide synthesizer, or a liquid handling robot.

In one embodiment, the system designs one or more promoter primers for each target nucleic acid. The system can optimize primer design for Tₘ, e.g., so all target binding regions of a group of promoter primers have a similar Tₘ, primer dimer formation, absence of palindromes, and so forth. The system can be interfaced with an oligonucleotide synthesizer to produce the promoter primers or oligonucleotide array synthesizer to produce an array of immobilized promoter primers.

In another aspect, the invention features a system that provides access to a database that includes information about transcript levels for a plurality of genes. The database can include records that include a reference describing a sample (e.g., tissue source, tissue type and so forth), a reference to a profile (the profile being a table describing transcript levels for the plurality of genes), and a locator indicating the identity or location of the support that includes archived templates that can be transcribed to produce aRNA or sRNA corresponding to the sample. The database can include at least ten records, e.g., each referring to a different mammalian tissue. In some embodiment, each sample is microdissected. In some implementations, the substrate can be provided to a user (e.g., a customer) in combination with access to the database, particularly to the record referring to that particular substrate. Database access can be provided in a variety of ways, e.g., by distribution of an access code (e.g., for Internet access) or by distribution of a machine readable medium that includes the records themselves.

Array Synthesis

Some embodiments use one or more arrays, for example: (1) an array of promoter primers; and (2) a detection array (e.g., a polymorphism or transcript detection array). An array can be a substrate that includes a plurality of addresses. Each address can include a homogenous population of immobilized nucleic acids, e.g., nucleic acids of predetermined sequence. The density of addresses can be at least 10, 50, 200, 500, 10³, 10⁴, 10⁵, or 10⁶ addresses per cm², and/or no more than 10, 50, 100, 200, 500, 10³, 10⁴, 10⁵, or 10⁶ addresses/cm². Addresses in addition to addresses of the plurality can be deposited on the array. The addresses can be distributed, on the substrate in one dimension, e.g., a linear array; in two dimensions, e.g., a planar array; or in three dimensions, e.g., a three dimensional array. (e.g., layers of a gel matrix).

In one embodiment, the substrate is an insoluble or solid substrate. Potentially useful insoluble substrates include: mass spectroscopy plates (e.g., for MALDI), glass (e.g., functionalized glass, a glass slide, porous silicate glass, a single crystal silicon, quartz, UV-transparent quartz glass), plastics and polymers (e.g., polystyrene, polypropylene, polyvinylidene difluoride, poly-tetrafluoroethylene, polycarbonate, PDMS, acrylic), metal coated substrates (e.g., gold), silicon substrates, latex, membranes (e.g., nitrocellulose, nylon). The insoluble substrate can also be pliable. The substrate can be opaque, translucent, or transparent. In some embodiments, the array is merely fashioned from a multiwell plate, e.g., a 96 or 384 well microtitre plate.

The array of promoter primers has an promoter primer at each address such that the promoter is accessible and functional and the target binding region is able to specifically recognize the target site. In some embodiments, the 3′ terminus of the promoter primer is extendable, e.g., by a DNA polymerase when hybridized to a template. The promoter primer can be anchored to the array substrate at the 5′ terminus. Alternatively, the promoter primer can be anchored to the array substrate at a non-terminal nucleotide, so long as the above preconditions are satisfied. In other embodiments, the 3′ terminus is non-extendable.

One method of anchoring promoter primers requires synthesizing an amino-modified nucleotide. During the phosphoramidite synthesis, at the desired position, an amino-modified nucleotide is included. The resulting amino-modified promoter primer is then deposited on a surface activated to covalent couple to amino groups. Such a surface and method are described in provisional patent application, U.S. Ser. No. 60/293,888, filed May 24, 2001. The surface is characterized by a covalently bonded activated group that includes an electron-withdrawing group on an N-substituted sulfonamide.

A second method of anchoring promoter primers requires synthesizing the promoter primers directly on a substrate using a 5′→3′ synthetic method, such as the method described in PCT US 01/02689. This method provides nucleotide arrays having C-5′ bound to the surface and C-3′ at the terminus. The arrays can be produced by reacting C-5′ activated, C-3′ photolabile group protected nucleotides, with a terminal hydroxyl group bound to the surface. After coupling a modified nucleotide to the surface, the C-3′ photolabile protecting group can be deprotected via a photochemical reaction to form a free hydroxyl group at the C-3′ terminus. The hydroxyl group, in turn, can react with a modified nucleotide including a C-5′ phosphorous activating group to tether the modified nucleotide to the surface. Repeated selective coupling of modified nucleotides carrying a C-5′ phosphorous activating group, such as phosphoramidite, and selective photodeprotection of the C-3′ photolabile protecting groups forms immobilized oligonucleotides arrays having C-5′ attached to the solid surface and the C-3′ at the terminal position. Selective photo-deprotection can be accomplished by several known methods, e.g. photolithography methods (as disclosed in Science (1991) 251:767-773; Proc. Natl. Acad. Sci. USA 93:13555-13560, (1996); U.S. Pat. Nos. 5,424,186; 5,510,270; and 5,744,305, and 5,744,101) or a digital micromirror technique (e.g., as described in Sussman et. al. (1999) Nature Biotechnology 17:974-97).

A third method of forming an SSP array includes the deposition of an unmodified oligonucleotide on a substrate. Numerous methods are available for dispensing small volumes of liquid onto substrates. For example, U.S. Pat. No. 6,112,605 describes a device for dispensing small volumes of liquid. U.S. Pat. No. 6,110,426 describes a capillary action-based method of dispensing known volumes of a sample onto an array.

In addition to these exemplary methods, any of the applicable array synthetic method can be used so long as the oligonucleotide is functional as a promoter and the target binding region is specific for the target site.

The second type of array includes a plurality of detection probes. The probes can be designed in any of a number of formats to detect SNPs or mRNA. For example, a pair of probes can be used for each biallelic SNP. Each pair has the appropriate nucleotide at the query position to detect one of the two alleles. The query position can be at the terminus of the detection probe. In another embodiment, the detection probe is a primer, and base extension protocols, e.g., as described in (Law and Brewer (1984) Proc. Natl. Acad. Sci. USA 81:66-70; Pastinen et al. (2000) Genome Res. 10: 1031-1042) are used to assess which allele is present. In still another embodiment, the query position is more centrally located, and the detection probe can be used, for example, as described in U.S. Pat. No. 5,968,740.

mRNA Libraries

One exemplary application of the invention is the amplification, analysis and archiving of mRNA populations. This process enables the high throughput amplification and detection of mRNA from small amounts of starting material, e.g., less than 1 μg, 100 ng, 10 ng, or 1 ng. For example, the process can be used to profile the expression of genes in a single cell. Further, the process results in an archive of the input nucleic acid sample. The archive can be repeatedly transcribed, to permit analysis of the sample. The mRNA population can be amplified using an immobilized oligonucleotide primer as a reverse transcription primer, e.g., as described above.

The benefits of the application are numerous. For example, the method does not require a number of manipulations such as precipitations and spin column separations. The washing and exchange of solutions is simplified as the cDNA archive is immobilized on the substrate. Washing also removes unbound targets, such as ribosomal RNA, which can interfere in reverse transcription by providing sites for non-specific priming.

The substrate serves as a DNA archive of the original mRNA sample. The archive can be returned to, time and again. Moreover, the archive is amplified by transcription, which restores the original sample in its RNA state. Such amplification is also linear and may be less susceptible to biasing events than, e.g., exponential amplification. In some embodiments, the method is supported by a single primer for reverse transcription. The primer is universal for all polyadenylated mRNAs.

In one embodiment, the method is used to archive an mRNA sample from a limited number of cells, e.g., fewer than 100 cells, e.g., a single cell. The DNA archive of the mRNA sample (or a sample of any nucleic acid) can be constructed for, e.g., normalized libraries, subtracted libraries and reduced complexity libraries.

RNA replicates generated from the substrate can be used, e.g., for profiling transcripts in the original mRNA sample, in vitro translating transcripts representative of transcripts in the original mRNA sample, and generating dsRNA.

In one embodiment, RNA replicates (aRNA or sRNA replicates, as appropriate) are used in a subtractive hybridization reaction. For example, aRNA replicates from a first sample can be subtracted from sRNA replicates produced from a second sample or from DNA produced from a second sample. Methods for subtractive hybridization are well known (e.g., one set of replicates can be attached to a substrate). In one embodiment, the method includes two subtraction hybridizations, one forward (e.g., aRNA vs. sRNA), the other backward (e.g., sRNA vs. aRNA). The net result is a highly differential comparison.

All cited references, patents, and patent applications are incorporated by reference in their entirety. Accordingly, U.S. Application Ser. Nos. 60/312,443, filed Aug. 15, 2001; 60/338,523, filed Nov. 5, 2001; 60/373,364, filed Apr. 16, 2002; Ser. No. 10/219,616 filed Aug. 15, 2002; and Ser. No. 10/341,199, filed Jan. 10, 2003, are incorporated by reference in their entirety. The following examples illustrate the specific embodiments of the invention described herein. As would be apparent to persons skilled in the arts, various changes and modifications are possible and are contemplated within the scope of the invention described.

### EXAMPLE 1

### Preparation of Aminopropylsilylated CPG Beads

10 g CPG beads (906 A, 80-128 mesh) were heated at 80° C. for 3 hr, cooled under nitrogen to the ambient temperature, and packed in a 25 mm×120 mm glass wool insulated HPLC column. 80 mL of 1:7 3-aminopropyltriethoxysilane and dry toluene were heated to around from 54° C. to 98° C. in a heated flask and then continuously pumped through the CPG beads packed column. The temperature of the column was monitored at around 37° C. to 45° C. during the course of reaction of about 38 hr. After the reaction was completed, the packed CPG beads were washed in the column twice with 125 mL methanol and twice with 125 mL acetone, and poured into a glass container. After drying under high vacuum in the glass container, the beads were stored under nitrogen ready for next synthesis.

### Preparation of 3-(3-(N-cyanoethyl-toluenesulfonamidocarbonyl)propyl-carboxamido-propylsilated CPG Beads

0.5 g of the above aminopropylsilylated CPG beads, 200 mg of 3-(toluenesulfonamido-carbonyl)propionic acid, 400 mg of Bop, and 4 mL dry NMP, and 0.25 mL diethylpropylamine were charged to a 6 mL reaction vial in a glove box. After shaking gently for about 3.5 hr, the CPG beads were transferred to a filtering cartridge, drained, and washed twice with 6 mL acetone, twice with 6 mL NMP, three times with 6 mL acetone. After drying under high vacuum, the beads were transferred to a 6 mL reaction vial. The beads were then treated with 4 mL dry NMP and 0.25 mL chloroacetonitrile and 0.25 mL diethylpropylamine, and shaken gently at the ambient temperature for about 21 hr. The CPG beads were transferred to a filtering cartridge, drained, and washed twice with 6 mL NMP, four times with 6 mL methanol, and twice with 6 mL acetone. After drying under high vacuum for 2 hr, the CPG beads were ready for solid phase covalent bonding reactions.

Experimental: CPG beads were activated. 100 pmols of AmC6N12T7dT20V oligo primers were coupled onto 4 mg of CPG beads in 400 mM sodium carbonate buffer at pH 9.5 for 1 hour at room temperature, followed by incubation overnight at 4° C. The beads were then blocked for two hours in blocking buffer, which contain 50 mM ethanolamine, 0.1 M Tris, and 0.1% SDS pH 9.0. After blocking, the beads were washed several times in Tris-buffered saline and either use immediately, or store in 70% ethanol. cDNA synthesis and transcription were carried out as described.

The sequence of [AmC6]N12T7dT20V: is as follows:

| | |
|---|---|
| 5′-[AmC6]ATAGGCGCGCCAATTAATACGACTCAC | (SEQ ID NO:28) |
| TATAGGGAGATTTTTTTTTTTTTTTTTTTTV-3′ | |

Results: Covalently coupled promoter primers, which contain a PEG linker and a 12 base spacer, were used successfully for template preparation and transcription from the CPG beads.

### EXAMPLE 2

### Covalent Coupling of TCR Primer to Microtitre Strips

TCR primer oligonucleotides with the following sequences were synthesized.

| | |
|---|---|
| N12 Primer: | |
| 5′-ATAGGCGCGCCAATTAATACGACTCACTATAGG | (SEQ ID NO:29) |
| GAGATTTTTTTTTTTTTTTTTTTTV-3′ | |
| N25 Primer: | |
| 5′-ACGTACGTACGTCATAGGCGCGCCAATTAATAC | (SEQ ID NO:30) |
| GACTCACTATAGGGAGATTTTTTTTTTTTTTTTTTT | |
| TV-3′ | |
| N50 Primer: | |
| 5′-ACGTACGTACGTACGTACGTACGTCACGTACGT | (SEQ ID NO:31) |
| ACGTCATAGGCGCGCCAATTAATACGACTCACTATA | |
| GGGAGATTTTTTTTTTTTTTTTTTTTV-3′ | |

Each oligonucleotide was phosphorylated at the 5′-end and purified by HPLC method individually. Each 5′-phosphorylated primer was then diluted to 100 nM in 10 mM 1-methylimidazole (pH 7.0) and the solution was added with 20 mM EDC. Each coating mixture was then pipetted to NucleoLink™ aminated strips (from Nalge Nunc International, Rochester, N.Y.): 100 μL per well. All the primer coupling reactions were continued at 42° C. for 4 hours. Each empty well was washed once at room temperature, soaked for 5 minutes at 42° C., and washed three more times at room temperature, all with 20 mM PBS (pH 7.4)/0.1% Tween 20/0.5% bovine y globulin (BgG.) To remove salt residues, the empty wells were washed three times with Milli Q water. The dry primer coated strips were stored at 4° C. until use.

To investigate the binding activity of TCR primer coated on the microtitre wells, samples of probe oligonucleotide of 5′-Bt-GCGCCAATTATCGAAAAAAAAAAAAAAAAAAAA (SEQ ID NO:32) were prepared in PBS/0.1% Tween 20/0.5% BgG, at the following concentrations: 0, 0.28, 0.56, 1.4, 2.8, 5.6, and 50 nM. The hybridization and detection assays were carried out in triplicates according to the following procedure.
1. Pipet 20 μL of biotinylated probe sample into each well.
2. Incubate at 42° C. for 30 minutes.
3. Wash 3 times with 100 μL of 2×SSC/0.1% Tween 20 buffer.
4. Pipet 20 μL of Streptavidin-horse radish peroxidase conjugate solution (Pierce Chemical), 0.2 μg/mL in PBS/0.05% Tween 20, into each well.
5. Incubate at room temperature for 30 minutes.
6. Wash once at room temperature, soak at 42° C. for 20 minutes, and then wash 3 more times, all with 100 μL of PBS/0.1% Tween 20/0.5% BgG.
7. Pipet 50 μL of 1.5 mM o-phenylenediame in 1× stable substrate (Pierce Chemical), into each well.
8. Incubate at room temperature for 15 minutes.
9. Add 150 μL of 0.5M Sulfuric acid to each well.
10. Measure absorbance at 492 nm on a microplate reader.

Results: The averaged absorbance from the triplicates of each sample is shown in the Table 2 below.

TABLE 2
Detection of Immobilized Oligonucleotides

| Probe Conc. | |
|---|---|
| (nM) | Absorbance |
| 0 | 0.015 |
| 0.28 | 0.835 |
| 0.56 | 1.303 |
| 1.4 | 1.708 |
| 2.8 | 2.094 |
| 5.6 | 2.224 |

This dose dependency of biotin probe concentration demonstrated a robust assay with 3 log-orders of dynamic range and also specific target binding activity for the primer immobilized on the substrate.

The microtitre strips coated with three different TCR primer oligonucleotides were used to amplify human liver RNA according to the following procedure.
1. 500 ng human liver RNA (Ambion, Inc. Austin, Tex.) was annealed to substrate anchored oligos in the presence of first strand synthesis buffer and DNase inhibitor was used in each of the two positive controls (1 μg per reaction.) The negative control was a well to which no RNA was added. mRNAs were annealed at 42° C. for 5 minutes.
2. cDNA synthesis was initiated by adding sodium pyrophosphate, and AMV reverse transcriptase. (Promega Catalog No. C4360)
3. Reactions were incubated at 42° C. for 1 hour.
4. Second strand cDNA synthesis was initiated by the addition of 40 μL of 2.5× second strand synthesis buffer (1×=40 mM Tris-HCl, pH 7.2); 5 μL of 1 mg/mL acetylated BSA; 23 units of DNA polymerase 1; 0.8 unit of RNase H, and nuclease free water to final volume of 100 μL. Incubated at 14-16° C. for 2 hours.
5. Wells were washed several times with 50 mM Tris-HCl, pH 8.0. The wells were then placed on ice, and 20 μL 1× T7 RNA polymerase transcription buffer was added.
6. Transcription reactions were performed in 20 μL volume, by following protocol provided by the manufacturer (AMPLISCRIBE™ T7 HIGH YIELD TRANSCRIPTION KIT, Cat No. AS2607, Epicentre, Madison, Wis.) Reactions were incubated at 37° C. for 1-2 hours.
7. 5 μL of the reactions was analyzed on an agarose gel.

Results: Agarose gel analysis revealed that RNA with a medium distribution between 0.4-1.0 kb was amplified by the N25 and N50 primers coated wells. N12 primer coated wells gave some amplification of RNA, but less than the amount by the N25 and N50 primers. See Table 3 below. No RNA transcripts were amplified by the negative controls.

TABLE 3

| | Purified RNA | Estimated Amplification |
|---|---|---|
| N12 Primer Coated Wells | 0.35 μg | 35 fold |
| N25 Primer Coated Wells | 1.91 μg | 191 fold |
| N50 Primer Coated Wells | 1.51 μg | 151 fold |

It was determined that the N25 primer coated wells repeatedly gave better amplification of RNA than N50 and N12 primers. The optimal length of the spacer is 25 nucleotides or a linker of equivalent length formed by a chemical substitute.

### EXAMPLE 3

### Biotinylated Primer Length

In these experiments, 5′ biotinylated oligonucleotide primers containing a 0, 6, or 12 nucleotides spacer sequence between the biotin and the promoter sequence, were tested for their effects on transcription. Reaction conditions for cDNA and for transcription were similar to previously described. Oligo-primer densities tested were 10 pmols per well and 1 pmol per well. The amount of RNA used was, variously, 1 microgram and 500 ng per reaction.

Enhanced amplification was observed with the primers that include 6 and 12 nucleotide spacers (Bt(6)T7dT20V and Bt(12)T7dT20V) relative to the amplification observed with the primer having a 0 nucleotide spacer (Bt(O)T7dT20V).

Primer Sequences:

| | |
|---|---|
| Bt(0)T7dT20V: | |
| 5′-Bt-ATTAATACGACTCACTATAGGGAGATTTTT | (SEQ ID NO:33) |
| TTTTTTTTTTTTTTTV 3′ | |
| Bt(6)T7dT20V: | |
| 5′-Bt-GCGCCAATTAATACGACTCACTATAGGGAG | SEQ ID NO:34 |
| ATTTTTTTTTTTTTTTTTTTTV 3′ | |
| Bt(12)T7dT20(V): | |
| 5′-Bt-ATAGGCGCGCCAATTAATACGACTCACTAT | (SEQ ID NO:35) |
| AGGGAGATTTTTTTTTTTTTTTTTTTTV-3′ | |

### EXAMPLE 4

The solution mode of sequence specific amplification by transcription was used to amplify the Stul fragment of the human apolipoprotein E gene. (This fragment codes for amino acid 72 to amino acid 209).

Human genomic DNA was purchased from Sigma Chemicals (St. Louis, Mo.). Samples of 20 μl containing 10 μg high molecular weight human DNA were digested for 3 hours at 37° C., using 10 units of Stul (New England Biolabs, Beverly, Mass.). The Stul digests were diluted to 50 μl in the presence of 1× lambda exonuclease buffer [67 mM Glycine-KOH(pH 9.4), 2.5 mM MgCl₂, and 50 μg/ml BSA], and 10 units of lambda exonuclease (New England Biolabs, Beverly, Mass.) and incubated at 37° C. for 30 minutes. This enzyme reaction was terminated by incubation at 75° C. for 30 minutes.

Subsequently, the reaction mixture was further diluted to 100 μl in the present of 1× Klenow fragment (3′→5′ exo⁻) buffer [10 mM Tris-HCl (pH 7.5), 5mM MgCl₂, 7.5 mM dithiothreitol], and 10 pmols of promoter primer (SEQ ID NO:1; T7StuSE), and hybridization was carried out at 37° C. for 10 minutes. The promoter primer anneals to one end of the StuI fragment of the human apolipoprotein E gene (amino acid 72 to amino acid 209). The following is the sequence of the T7StuSE:

| | |
|---|---|
| 5′-AATTAATACG ACTCACTATA GGGAAGGCCT | (SEQ ID NO:1) |
| ACAAATCGGA ACTGGAG-3′ | |

The T7 polymerase promoter is underscored. The apoE annealing site is 3′ to the promoter.

After promoter primer annealing, the primer and apoE target are extended by the addition of 10 units of Klenow fragment DNA polymerase (New England Biolabs, Beverly, Mass.), 10 mM each of dATP, dGTP, dTTP, and dCTP during incubation at 37° C. for 1 hour. After heat inactivation of the enzyme at 75° C. for 30 minutes, the mixture was adjusted to 2.5 M ammonium acetate, and two volumes of 100% ethanol were added to precipitate the DNA. DNA was then recovered by centrifugation and dissolved in 20 μl of 10 mM Tris-HCl at pH 8.0.

The apoE target was then amplified by transcription. An aliquot of the ethanol precipitated DNA was in vitro transcribed using the AMPLISCRIBE™ T7 transcription kit from Epicentre (Madison Wis.). The resulting transcription products were analyzed by agarose gel electrophoresis. RNA products of the expected size were observed only in promoter primer extended genomic DNA, and were absent in controls from unprimed genomic DNA.

Gel electrophoresis of the following samples validated the method. Lane 1: 100 bp DNA marker; Lane 2: 10% of the T7 transcription reaction from 250 ng of lambda exonuclease treated, human genomic DNA; Lane 3: 10% of the T7 transcription reaction from 250 ng of lambda exonuclease treated, SSPP primer extended human genomic DNA; Lane 4: 10% of the T3 transcription reaction from same DNA as in Lane 3; Lane 5: 10% of the T7 transcription reaction from 60 ng of clone apoE DNA and treated as in Lane3; Lane 6: 10% of the T3 transcription reaction from same DNA as in Lane 5; Lane 7: 10% of the T7 transcription reaction from apoE clone, no treatment; Lane 8: 1 μg human genomic DNA, no treatment

To confirm that in-vitro transcribed RNA is indeed apoE RNA, RT-PCR was performed according to the protocol provided by the vendor (THERMOSCRIPT™ RT PCR systems, Life Technologies, Bethesda, Md.). PCR using the primer pair, P3 and P6ASE (SEQ ID NO:2 and SEQ ID NO: 3), produced the correct size product, only for DNA derived from RNA transcribed from promoter primer-extended genomic DNA. There is no PCR product derived from the RNA transcription mixture, suggesting the PCR product is not from unprimed genomic DNA. There is no PCR product when using the primer pair T7 and P6ASE (SEQ ID NO:4 and SEQ ID NO:3) confirming the PCR template is indeed cDNA derived from RNA.

Gel electrophoresis of controls and test reactions validated method. A specific amplified fragment was evident when human genomic DNA was used as the template with the appropriate primers. The amplified RNA was detected by PCR. Further, he PCR product was isolated from preparative agarose gel electrophoresis, and sequenced. DNA sequencing confirmed that the PCR product was indeed apoE.

These reactions and manipulations can be coupled and streamlined to achieve considerable gains in efficiency and economy.

### EXAMPLE 5

SSAT is suited for multiplex reactions. In this example, multiple target fragments were amplified by site specific amplification by transcription. A 5.5 kb genomic human apoE serve as DNA target template in this manipulation. Briefly, apoE DNA is cleaved by restriction enzymes Aval, BsrD1, and StuI (New England Biolabs, Beverly, Mass.) to generate eight DNA fragments. One of the eight DNA fragment is the same Stul fragment as described previously in Example 1. The human apoE sequence is available, e.g., from GenBank entry AF 261279.

The primer sequences listed in SEQ ID NOS:1 to 22 were used:

SEQ ID NO:1 (T7StuSE):
AATTAATACG ACTCACTATA GGGAAGGCCT ACAAATCGGA
ACTGGAG
SEQ ID NO:2 (P3):
GAACAACTGA CCCCGGTGGC GG
SEQ ID NO:3 (P6ASE):
GAGGCGAGGC GCACCCGCAG
SEQ ID NO:4 (T7):
TTAATACGAC TCACTATAGG G
SEQ ID NO:5 (T7AvaSE2):
CATTAATACGACTCACTATAGGGACTCGGGGTCGGGCTTGGGGAGA
SEQ ID NO:6 (T7AvaSE3):
CATTAATACGACTCACTATAGGGACCCGGGAGAGGAAGATGGAATTTTC
SEQ ID NO:7 (T7AvaSE4):
CATTAATACGACTCACTATAGGGACCCGAGCTGCGCCAGCAGACCGAG
SEQ ID NO:8 (T7BsrD1SE):
CATTAATACGACTCACTATAGGGACATTGCAGGCAGATAGTGAATACC
SEQ ID NO:9 (T7stuSE2):
CATTAATACGACTCACTATAGGGAAGGCCTGGGGCGAGCGGCT
SEQ ID NO:10 (T7StuSE3):
CATTAATACGACTCACTATAGGGAAGGCCTTCCAGGCCCGCCTCAAGA
SEQ ID NO:11 (AvaSE2):
CTCGGGGTCGGGCTTGGGGAGA
SEQ ID NO:12 (AvaSE3):
CCCGGGAGAGGAAGATGGAATTTTC
SEQ ID NO:13 (AvaSE4):
CCCGAGCTGCGCCAGCAGACCGAG
SEQ ID NO:14 (BsrD1SE):
CATTGCAGGCAGATAGTGAATACC
SEQ ID NO:15 (StuSE2):
AGGCCTGGGGCGAGCGGCT
SEQ ID NO:16 (StuSE3):
CCTTCCAGGCCCGCCTCAAGA
SEQ ID NO:17 (AvaASE2):
CCCAGTAGGTGCTCGATAAATG
SEQ ID NO:18 (AvaASE3):
AGAAGAGGGGGCCCAGGGTCTG
SEQ ID NO:19 (AvaASE4):
TGAGTCAGAAGGGAAGAGAGAGAG
SEQ ID NO:20 (BsrD1ASE):
AGCACAGGTGTGTGGCACCATG
SEQ ID NO:21 (StuASE2):
CTCGTCCAGGCGGTCGCGGGT
SEQ ID NO:22 (StuASE3):
TCCACCCCAGGAGGACGGCTG

10 μg of a plasmid DNA containing the 5.5 kb human apoE gene was digested with 40 units of Aval and 40 units of Stul for 4 hours at 37° C. Subsequently, 20 units of BsrD1 was added to the reaction mixture. Incubation was continued for an additional 2 hours at 65° C. The restriction digestion was quenched on ice. apoE DNA fragments were purified by the mini-elute enzyme clean-up kit (QIAGEN Inc.). An aliquot of 2 μg of the restricted DNA was treated with 2 units of lambda exonuclease at 37° C. for 30 minutes. The exonuclease reaction was terminated and inactivated by incubation at 80° C. for 15 minutes. The reaction mixture was adjusted to contain 2.5 M ammonium acetate, and precipitated by the addition of 2.5 volumes of 100% ethanol. The resulting mixture was then incubated on ice for two hours, and then centrifuge at room temperature at 16,000×g for 15 minutes in a Beckman Allegra micro-centrifuge to pellet the DNA. The ethanol supernatant was removed by pipetting, and the DNA pellet was rinsed with 70% ethanol, air dried, and dissolve in sterile water.

Primer annealing was carried out in 30 μl containing 1× Klenow (3′-5′exo⁻) buffer, 50 pmols of each T7apoE sequence primers (SEQ ID NO: 1, 5, 6, 7, 8, 9 and 10) and 1.8 μg of the lambda exonuclease treated DNA. The reaction mixture was heated at 75° C. for 5 minutes, followed by incubation at 37° C. for another 10 minutes. The annealing mixture was then diluted to 50 μl 1 with 1× Klenow buffer in the presence of 1 mM dNTPs and 10 units of Klenow enzyme (3′-5′ exo⁻) for 1 hour at 37° C. The extension reaction was terminated by heating at 75° C. for 20 minutes to inactivate the enzyme. Excess T7apoE sequence primers were removed using Exonuleasel (Amersham Pharmacia Inc.). Exonucleasel was removed by the mini-elute enzyme purification kit as described earlier.

To detect the promoter primer-extended product, an aliquot of the treated DNA was in vitro transcribed using the AMPLISCRIBE™ T7 transcription kit from Epicentre, (Madison, Wis.). The RNA was then reverse transcribed into cDNA using antisense apoE primers specific for each of the seven restriction fragments (SEQ ID NO:3, 17, 18, 19, 20, 21 & 22, respectively). Only the RNA which includes the corresponding sense strand of the apoE reverse transcription primer sequences were reverse transcribed into cDNA. cDNA synthesis reaction was carried out according to the protocol of the THERMOSCRIPT™ RT PCR system (Life Technologies, Bethesda, Md.). PCR reactions were carried out in 20 μl volume, in an Eppendoff DNA thermocycler, using AMPLITAG™.

Gold DNA polymerase and apoE sequence primer pairs which are specific for the seven target restriction fragments (SEQ ID NO:2,3, 11-22.). A PCR assay detected amplified products from all RNA-amplified apoE fragments: AvaI-2, AvaI-3, AvaI-4, BsrD1, StuI-1, StuI-2, and StuI-3. All seven primer pairs amplified a prominent band of the expected size from cDNA but not from RNA. DNA sequencing of five representative fragments confirmed that they are all correct apoE sequences.

### EXAMPLE 6

### Solid Phase Based Amplification

This example illustrates solid-phase sequence specific amplification by transcription. The Stul fragment of human apolipoprotein E gene (encoding amino acid 72 to amino acid 209) is the test substrate model. The following primer sequences were used: SEQ ID NO: 2, SEQ ID NO: 3, and SEQ ID NO: 23.

Streptavidin coated microplates (NoAb BioDiscoveries, Mississauga, Ontario, Canada) were used to immobilize 5′ end biotin labeled, promoter primer oligonucleotide (BT7StuSE1). Briefly, oligos were diluted to 100 μl with TBS (Tris-Buffered Saline, 20 mM Tris, 500 mM sodium chloride, pH 7.5), and spotted into 8-well strips. Each well contained either 50 pmols or 5 pmols of BT7StuSE1. The negative-control well contained 50 pmols of T7StuSE1 oligo (SEQ ID NO: 1) primer. After a two hour incubation at room temperature, oligonucleotide solutions were discarded, and the wells were rinsed several times with 100 μl TBS, 0.01% Tween 20, followed by incubation at room temperature for 30 minutes with a blocking solution of TBS, 0.01% Tween 20 and 100 μg/ml BSA (New England Biolabs, Beverly, Mass.). The wells were then rinse twice, with 100 μl TBS followed by 100 μl of Klenow buffer (3′-5′ exo⁻). Single-stranded DNA were created by lambda exonuclease digestion of the restricted human Apo E fragments, as previously described in examples 1 and 2. 1 μg of the single-stranded DNA was annealed to the immobilized primers for 15 minutes at 37° C. in 30 μl of Klenow buffer, followed by primer-extension in 100 μl reaction volume that contained Klenow buffer, 1 mM dNTPs, and 20 units of Klenow DNA polymerase (3′-5′ exo⁻). Enzyme incubation was for one hour at 37° C. in a humidity chamber. The wells were then washed several times with 200 μl of sterile 10 mM Tris-HCl, pH 8.0, and once, with 50 μl 1× T7 RNA polymerase transcription buffer. In-vitro transcription was carried out in 20 μl at 37° C. for two hours using the T7 AMPLISCRIBE™ kit from Epicentre Technologies (Madison, Wis.). The resulting transcription products were analyzed by agarose gel electrophoresis. RNA product of the expected size was observed only in wells, which contained immobilized Bt7StuSE1 primer, and is absent from the well which contained the T7Stul oligonucleotide primer. Furthermore, repeatable, robust transcription was maintained over a period of several days of storage at 4° C. After an initial round of transcription, the substrate was stored at least for 24 hours, and then removed for further transcription. Five such reactions over a storage period of ten days provided continued amplification with no loss in yield.

To confirm the in-vitro transcribed RNA is indeed apoE RNA, RT-PCR was performed according to the protocol provided by THERMOSCRIPT™ RT PCR systems (Life Technologies, Bethesda, Md.). To confirm the PCR product is indeed apoE sequence, the PCR product was isolated from preparative agarose gel electrophoresis and sequenced. DNA sequencing confirmed the PCR product was indeed apoE.

### EXAMPLE 7

Human whole genomic DNA was treated with lambda exonucleases, hybridized to SSP primers attached to a substrate, extended using a DNA polymerase, then amplified in accordance with the single promoter SP-TCR method. Robust transcription was observed using input human genomic DNA in an amount between 100 ng and 2 μg. The detection of RNA amplified transcripts from the 10 ng sample is indicative of the unexpected amplification yield provided by the method. No RNA amplified products were detected in a negative control reaction. The reaction product migrated as a discrete band on agarose gel.

### EXAMPLE 8

Materials for this example included:
mRNA: Human liver Poly A RNA purchased from Ambion Inc., Austin, Tex.
Anchor primer: Bt-T7d(T)₁₇V where Bt=5′biotin; T7=T7 RNA polymerase promoter; d (T) 17=a homopolymer of 17 T residue; V=A, G, and C. This primer has the sequence: 5′-TTAATACGACTCACTATAGGGTTTTTTTTTTTTTTTTTV-3′ (SEQ ID NO:26)
Solid phase: streptavidin coated wells (NoAb Biodiscoveries, Mississauga, Ontario, Canada)

The procedure was as follows:
1. 200 pmol Anchor Primer was attached each streptavidin coated well,
2. Wells were washed with TBS and rinsed with 1× first strand synthesis buffer,
3. 2 μg human liver mRNA (Ambion, Inc. Austin, Tex.) was annealed to anchored oligos in the presence of first strand synthesis buffer, and DNase inhibitor (UNIVERSAL RIBOCLONE™ cDNA Synthesis System Catalog No. C4360, Promega Corp, Madison, Wis., USA). Kanamycin mRNA(Promega Corp) was used as a positive control (1 μg per reaction). The negative control was a well to which no RNA was added. mRNAs were annealed at 42° C. for 5 minutes.
4. cDNA synthesis was initiated by adding sodium pyrophosphate, and AMV reverse transcriptase (All reagents from Promega Catalog No. C4360). The final concentrations for all the components were: 1× first strand synthesis buffer (50 mM Tris-HCL, pH 8.3 at 42 degree C.; 50 mM KCL; 10 mM MgCl2; 0.5 mM spermidine; 10 mM DTT; 1 mM each dATP, dCTP, dGTP, dTTP); 40 units of Rnasin ribonuclease inhibitor; 4 mM sodium pyrophosphate and 30 units of AMV reverse transcriptase. The final volume of first strand cDNA synthesis reaction was 20 μl.
5. Reactions were incubated at 42° C. for 1 hour.
6. Second strand cDNA synthesis was initiated by the addition of 40 μl of 2.5× second strand synthesis buffer (1×=40 mM Tris-HCL, pH 7.2); 5 μl of 1 mg/ml acetylated BSA; 23 units of DNA polymerase 1; 0.8 unit of RNase H, and nuclease free water to final volume of 100 μl. Incubate at 14-16° C. for 2 hours.
7. 2 units of T4 DNA polymerase/μg of input RNA was added. Incubation was continued at 37° C. for 10 minutes.
8. Wells were washed several times with 50 mM Tris-HCL, pH 8.0. The wells were placed on ice, and 20 μl 1× T7 RNA polymerase transcription buffer was added.
9. Transcription reactions were performed in 20 μl volume, by following protocol provided by the manufacturer (AMPLISCRIBE™ T7 high yield transcription kit, Cat#AS2607, Epicentre, Madison, Wis.). Reactions were incubated at 37° C. for 1-2 hours.
10. 5 μl of the reactions was analyzed on an agarose gel.

Results: A distribution of nucleic acid fragments corresponding to RNA transcripts of >0.4 kb, with a medium distribution between 0.4-1.0 kb were observed in both reaction primed by human liver mRNA primed cDNA library, and the reaction primed by kanamycin resistance gene mRNA. No RNA transcripts were detected from the negative control.

Nucleic acid was amplified by RT-PCR from the transcription reaction product produced from the insoluble substrate. Specific size fragments corresponding to mRNAs for human serum albumin, beta-actin, and G3PDH were detected in the sample derived from the human liver mRNA sample, but not from the control sample of mRNA for the kanamycin resistance gene. Similarly, a nucleic acid fragment for the kanamycin resistance gene was detected in this control, whereas the liver specific transcripts were not. This example demonstrated that mRNA can be amplified from a substrate prepared as described.

### EXAMPLE 9

Materials for this example included:
mRNA: Human liver total RNA, and yeast RNA (Ambion Inc., Austin Tex.). Kanamycin resistance gene control mRNA (Promega Corp).
Anchor primers: 1) Bt-T7d(T)₁₇V (see above). 2) Bt-ASC 1T3 where Bt=5′ biotin; T3=T3 promoter sequence; ASC1=restriction endonuclease recognition site for AscI (GGCGCGCC). 3) TCR-adapter
Solid phase: streptavidin coated wells (NoAb Biodiscoveries, Mississauga, Ontario, Canada)

The first part of the procedure was as follows:
1. 200 pmol Anchor Primer was attached each streptavidin coated well,
2. Wells were washed with TBS and rinse with 1× first strand synthesis buffer,
3. Samples were annealed to anchored oligos in the presence of first strand synthesis buffer, and DNase inhibitor (UNIVERSAL RIBOCLONE™ cDNA Synthesis System Catalog No. C4360, Promega Corp, Madison Wis.). Four separate reactions were set up. The reaction samples were: (a) 20 μg human liver total RNA; (b) 20 μg human liver total RNA+1 ng kanamycin mRNA; (c) 20 μg human liver total RNA+10 ng kanamycin mRNA; and (d) 20 μg yeast RNA+100 ng kanamycin mRNA K. mRNAs were annealed at 42° C. for 5 minutes.
4. cDNA synthesis was initiated by adding sodium pyrophosphate, and AMV reverse transcriptase (all reagents from Promega Catalog No. C4360). The final concentrations for all the components were: 1× first strand synthesis buffer (50 mM Tris-HCL, pH 8.3 at 42 degree C.; 50 mM KCL; 10 mM MgCl2; 0.5 mM spermidine; 10 mM DTT; 1 mM each dATP, dCTP, dGTP, dTTP); 40 units of Rnasin ribonuclease inhibitor; 4 mM sodium pyrophosphate and 30 units of AMV reverse transcriptase. The final volume of first strand cDNA synthesis reaction was 20 μl.
5. Reactions were incubated at 42° C. for 1 hour.
6. Second strand cDNA synthesis was initiated by the addition of 40 μl of 2.5× second strand synthesis buffer (1×=40 mM Tris-HCL, pH 7.2); 5 μl of 1 mg/ml acetylated BSA; 23 units of DNA polymerase 1; 0.8 unit of DNase H, and nuclease free water to final volume of 100 μl. Incubate at 14-16 degree C. for 2 hours.
7. 2 units of T4 DNA polymerase/μg of input RNA were added. Incubation was continued at 37° C. for 10 minutes.
8. Wells were washed several times with 50 mM Tris-HCL, pH 8.0.
9. TCR-Adapter ligation: Adapter ligation was performed in 30 ul volume using the FAST-LINK DNA™ ligation kit (Epicentre Cat#LK0750H, Madison Wis.). The final concentration of all the components were: 1× ligation buffer (33 mM Tris-acetate pH 7.8, 66 mM potassium acetate, 10 mM magnesium acetate, 5 mM DTT); 05 mM ATP; 20 pmol TCR-adapter. Incubate at room temperature for 30 minutes, and wash well several times with 50 mM Tris-HCL pH 8.0 followed by 20 μl of 1× T7 transcription buffer.
10. Transcription reactions were performed in 20 μl volume following the protocol provided by the manufacturer (AMPLISCRIBE™ T7 high yield transcription kit, Cat#AS2607, Epicentre, Madison, Wis.). Reactions were incubated at 37° C. for 1-2 hours.
11. 4 μl of each reaction were analyzed on an agarose gel.

Result: The following RNA amplification products were observed: RNA transcripts >0.4 kb in length, with a medium distribution between 0.4-1.0 kb were observed in all reactions that amplified human liver total RNA spiked with 0, 1, or 10 ng of kanamycin mRNA), and a discrete RNA band was observed in reaction #4 which is derived from the control mRNA for the kanamycin resistance gene.

These findings indicate that a cDNA library can be synthesized on an insoluble substrate from 20 μg of input human total RNA (which corresponds to approximately 200-400 ng of mRNA). The library can be amplified by transcription.

An individual species of 1 ng or less can be detected by this method as demonstrated by detection of the spiked RNA for the kanamycin resistance gene. Further, the library was stored for several days under refrigeration. The stored library was effectively transcribed, thus verifying the value of this technique for archiving RNA populations. It was also found that the stored library could be effectively transcribed after two or more months of storage.

### EXAMPLE 10

RNA (16 μl) from the first T7 transcription (Example 6) was precipitated with ethanol and redissolved in 20 μl of nuclease-free water. 4 μl of RNA were used for T3 transcription cycling (TCR). The experimental procedures were as follows:
1. 200 pmol of the Bt-T3ASC1 oligonucleotide was attached to streptavidin coated wells
2. Wells were washed with TBS and rinsed with 1× first strand synthesis buffer
3. The mRNA was annealed to the anchored oligonucleotides in the presence of first strand synthesis buffer, and DNase inhibitor. A total of 4 reactions were set-up as follows:
   a) 4 μg RNA from T7 reaction 1 of Example 6;
   b) 4 μg RNA from T7 reaction 2 of Example 6
   c) 4 μg RNA from T7 reaction 3 of Example 6
   d) 4 μg RNA from T7 reaction 4 of Example 6
   Annealing was performed at 42° C. for 5 minutes.
4. cDNA synthesis was initiated by adding sodium pyrophosphate, and AMV reverse transcriptase. The final concentrations for all the components was: 1× first strand synthesis buffer (50 mM Tris-HCL, pH 8.3 at 42° C.; 50 mM KCL; 10 mM MgCl₂; 0.5 mM spermidine; 10 mM DTT; 1 mM each dATP, dCTP, dGTP, dTTP); 40 units of Rnasin ribonuclease inhibitor; 4 mM sodium pyrophosphate and 30 units of AMV reverse transcriptase. The final volume of the first strand cDNA synthesis reaction was 20 μl.
5. The reaction was incubated at 42° C. for 1 hour.
6. Second strand cDNA synthesis was effected by the addition of 40 μl of 2.5× second strand synthesis buffer (1×=40 mM Tris-HCL, pH 7.2); 5 ul of 1 mg/ml acetylated BSA; 23 units of DNA polymerase 1; 0.8 unit of DNase H, and nuclease free water to final volume of 100 μl. Incubate at 14-16° C. for 2 hours.
7. Then 2 μl (6 units) of E. coli ligase was added to each well, and the incubation was extended for 30 minutes at 16° C.
8. 2 units of T4 DNA polymerase/μg of input RNA was added to each well, and the incubation was extended for 10 minutes at 37° C.
9. After the incubation, wells were washed several times with 50 mM Tris-HCL, pH 8.0.
10. Then transcription was used to amplify RNA from each well. Transcription reactions were performed in 20 μl following the protocol of AMPLISCRIBE™ T3 High Yield Transcription Kit, (Cat#AS2603, Epicentre, Madison Wis.). Reactions were incubated at 37° C. for 1-2 hours.
11. The reaction was analyzed by agarose gel electrophoresis.

Results: Transcription products having a size of 0.4-1 kb were observed in all 4 reactions. These results demonstrated the TCR adapter ligated to the ends of the T7d(T)₁₇V primed cDNA from Example 9 was effective for driving T3 DNA polymerase mediated amplification. This example is a successful application of the so-called Transcription Chain Reaction (TCR) method.

The incorporation of a rare sequence cutter restriction enzyme such as AscI (which cuts human DNA on average once per 670,000 base pairs) permitted the release of the anchored library cDNA from the substrate, thereby providing flexibility in downstream applications. It was also found that three full cycles of TCR amplification had an amplification power of greater than 10⁸, and that 1 ng of total RNA was successfully amplified.

### EXAMPLE 11

An exemplary dG tailing procedure is as follows:
1. Immobilize Bt12T7d(T)20V anchor primer
2. Anneal total RNA
3. First strand synthesis as described
4. Rinse three times with 50 mM Tris-HCL (pH 8.0), followed by rinsing with terminal transferase buffer.
5. Add terminal transferase buffer, 25 μM dGTP, 10 units of terminal transferase in 20 μl reaction, and incubate at 37° C. for 15 minutes
6. Wash wells and proceed to Tag annealing

Tag annealing and second strand synthesis includes: combining 20 pmol Tag (LITP-1) in 20 μl EcoPoll buffer (New England Biolabs, Beverly, Mass.), incubating at 37° C. for 10 minutes; adding dNTPs to final 1 mM; 5 ug nuclease free BSA; 0.8 units of RnaseH, and 1 unit of Klenow enzyme in final volume of 50 μl, and incubating for 1 hour at 37° C.

Transcription of the immobilized template produced by this method is as described.

### EXAMPLE 12

### Amplification of Target Nucleic Acids

This example uses solid phase transcription chain reaction (SPTCR).

A synthetic RNA produced from a cassette that includes a gene encoding kanamycin resistance and an engineered poly (A) sequence at the 3′ end of the RNA (Promega Inc. Wis.) was used as a target. We have observed that 10 pg of this kan target mRNA can be amplified by SP-TCR using the Klenow enzyme.

We used varying amounts of this synthetic RNA as an input for SPTCR amplification reactions. The amounts were 10 fentograms (fg), 100 attograms (ag), 10 ag and 5 ag. Amplifications were performed using two different types of promoter primters:
(a) 5′ biotin-N12-T7-oligod(T)20V, and
(b) BT7kan22R which includes a sequence complementary to nucleotides 981-960 of the kan resistance gene.
1. Promoter-Primer binding: 1 pmol of the 5′ biotin-N12-oligod(T)20V or the 5′ biotin-N12-kan-22R Primer (NT 981-960) was added to each streptavidin coated microtiter well in 20 ul of TBS ( 20 mM Tris·HCl, 500 mM NaCl, pH 7.5), and binding proceed at room temperature for two hours. The wells were rinsed several times with 50 mM Tris-HCl pH 8.0 to remove any unbound primers.
2. Target capture: Kan mRNA of various amounts as described above were added to each well in the presence of 1× first strand buffer (50 mM Tris-HCl, pH8.2, 10 mM MgCl₂, 50 mM KCl, 0.5 mM spermidine, and 10 mM DTT), and 20 units of SUPERASE-IN™ Rnase inhibitor, in a final volume of 14.5 μl. The target capture or annealing reaction was for 10 minutes at 42° C. with gentle shaking.
3. First strand cDNA synthesis: At the end of 10 minutes annealing, 5.5 ul of first strand reaction mix, which contains 30 units of AMV-RT, 4 mM sodium pyrophosphate, and 1 mM of dNTP was added, and incubate at 42 degrees C. for 1 hour with gentle shaking.
4. Second strand synthesis: Second strand synthesis was carried out in 100 ul in the presence of 1× Klenow buffer (50 mM Tris-HCl pH 7.2, 10 mM MgCl₂, and 0.1 mM DTT), 5 ug of acetylated BSA, 0.8 unit of RnaseH, and 10 units of Klenow DNA polymerase, or E. coli DNA polymerasel at 37 degree C. with gentle shaking for 2 hours. The solutions were then removed, and each well was rinsed three times with 50 mM Tris-HCl, pH 8.0.
5. Transcription: T7 RNA polymerase reactions were carried out in 20 ul volume as described by manufacturers (Ambion MEGASCRIBE™ kit)

The capture and amplification of target kan mRNA was assayed by RTPCR and compared to PCR results of known kan cDNA. The results showed that SPTCR can capture and amplify as little as 5 ag (8.5 copies) of target (kan) mRNA. When compared to kan cDNA PCR standards, the fold amplification was: 1,600 (the BT7kan22R primer), and 16,000 (the B-N12-T7-d (T) 20V primer).

### EXAMPLE 13

### Amplification in the Presence of a Capture Probe and Detection Probe

This example evaluates the use of SPTCR in the presence of a capture probe. The capture probe had a design suitable for selectively hybridizing to and immobilizing the amplified strand of a target nucleic acid. In this example we demonstrated the presence of excess amount of capture probe and the detection probe did not prevent the detection of kan mRNA. In the first experiment, we used Bt-N12-T7-oligod(T) 20V anchor primer, and 10 ng in-put kan mRNA in each SPTCR reaction. Eight reactions were performed. Two reactions contained 2 pmol of each capture probe, kancap747 (5′-biotin TAATAGGTTGTATTGATGTTGGACGAGTCGGAATCGCAGAC, dCrev-Q nt747-788, SEQ ID NO:36), and detection probe KanDet801 (5′ Cy3 TTGCCATCCTATGGAACTGCCTCGGTGAGT, nt801-830, SEQ ID NO:37), added during cDNA synthesis. Another two reactions contained 2 pmol of the capture probe, but the detection probe was added, after cDNA synthesis, during the T7 transcription step. The remaining four reactions contained no added capture probe and detection probes. The amount and profile of the amplified RNA were very similar in all eight reactions. Therefore, the capture probes and the detection probes did not interfere with SPTCR reactions. The presence of capture primer did not prevent the detection of as little as 5 ag (8.5 copies) of target kan mRNA.

### EXAMPLE 14

In this example, we evaluated use of SPTCR to detect an RNA species (in this example, kan mRNA) in the presence of human saliva.

10 μl of saliva was used in place of nuclease free water and compared to controls which included no saliva, but nuclease free water instead. 1 pg or 1 fg input Kan mRNA was added as the target nucleic acid. The Rnase inhibitors used include various combinations of: SuperaseIn, RIP, and Rnase inhibitor III. One of the combinations of inhibitors included all three of the inhibitors. The kan mRNA were exposed to the saliva for 30 minute during the annealing step. The rest of the SPTCR protocol was identical to the above experiments. The results showed that target mRNA could be detected by SPTCR despite the presence of human saliva. The results also indicated that a combination of Rnase inhibitors may be more effective than a single inhibitor.

### EXAMPLE 15

This example includes the following steps:
a) Immobilization of a mixture of the first and second oligomers. Properties of the two oligomers have been described above.
b) The sample containing a specific RNA or DNA as a first template is in contact with the solid surface where the primer sequence of the immobilized first oligomer can recognize and extract the RNA through hybridization.
c) Primer extension using the RNA or DNA as the template to form a cDNA is performed to form a RNA:DNA or DNA:DNA hybrid through the use of reverse transcripts, or DNA polymerase.
d) RNA in the RNA:DNA hybrid is nicked with Rnase H
e) Double-stranded cDNA is formed using a DNA polymerase. For example, a DNA polymerase without 5′ exonuclease activity, such as Klenow fragment, is used.
f) T7 RNA polymerase initiates transcription at the promoter to synthesize a plurality of copies of antisense RNA from the double-stranded cDNA.
g) The RNA produced during the transcription is captured by the neighboring immobilized second oligomer through hybridization.
h) The captured RNA is detected by hybridization with a third oligomer, which contains a detectable label, e.g., a label that can produce a signal.

Although the above events occur in sequential steps, the reagents, which are needed for the processes can be added either in sequential steps or as a mixture. The temperatures used are mostly held at constant. The reagents involved includes a reverse transcriptase, such as AMV; Rnase H; Klenow or DNA polymerase 1; T7, or other RNA polymerases; deoxyribonucleotide triphosphates; ribonucleotide triphosphates; Rnase inhibitors, and sodium pyrophosphate.

A number of embodiments of the invention have been described. Nevertheless, it will be understood that various modifications may be made without departing from the spirit and scope of the invention. Accordingly, other embodiments are within the scope of the following claims.

1. A substrate that comprises:
a promoter primer that can be extended to form a transcribable template nucleic acid; and
a capture probe, wherein the promoter primer and the capture probe are non-complementary, and the capture probe can specifically bind to a target nucleic acid or complement thereof.

2. The substrate of claim 1 wherein the substrate is planar.

3. The substrate of claim 1 wherein the capture probe is exactly complementary to the target nucleic acid.

4. The substrate of claim 1 wherein the capture probe is un-extendable.

5. The substrate of claim 1 wherein the capture probe is attached to the substrate by its 3′ end.

6. The substrate of claim 1 wherein the 3′ end of the capture probe lacks a terminal hydroxyl.

7. The substrate of claim 1 wherein the promoter primer comprises a polyT sequence that can anneal to the 3′ end of eukaryotic mRNAs.

8. The substrate of claim 7 wherein the polyT sequence terminates with a mono or dinucleotide other than T at the 3′ most region of the promoter primer.

9. The substrate of claim 1 wherein the promoter primer comprises a target specific sequence that can anneal to a target RNA or DNA sequence.

10. The substrate of claim 1 wherein the promoter primer comprises a sequence that is recognized by a prokaryotic RNA polymerase to initiate transcription.

11. The substrate of claim 1 further comprising a microchannel that places a region of the substrate occupied by the promoter primer in fluid communication with a region occupied by the capture probe.

12. A method comprising:
providing the substrate of claim 1;
contacting a sample to the substrate;
forming template nucleic acids by extending the promoter primer;
transcribing the template nucleic acids, thereby providing transcripts; and
evaluating binding of the transcripts to the capture probe.

13. The method of claim 12 wherein the promoter is a prokaryotic promoter.

14. The method of claim 12 wherein the transcribing is effected in the presence of a labeled ribonucleotide that can be incorporated into the transcripts.

15. The method of claim 12 wherein the evaluating comprises detecting presence of the labeled ribonucleotides incorporated into the transcripts that bind to the capture probe.

16. The method of claim 12 wherein the evaluating comprises contacting a detector probe to the substrate, wherein the detector probe can specifically anneal to a region of the target nucleic acid that does not overlap with the capture probe.

17. The method of claim 12 wherein the detector probe comprises a label.

18. A substrate that comprises:
immobilized template nucleic acids that comprise a promoter region;
one or more RNA polymerases that can collectively transcribe the immobilized template nucleic acids, and
a capture probe that can hybridize to a target nucleic acid, if present.

19. The substrate of claim 18 further comprising a detector probe.

20. The substrate of claim 19 wherein the capture probe is immobilized, but the detector probe is present in a diffusable form on the substrate.

21. The substrate of claim 18 wherein the RNA polymerases are provided in a crystalline form.

22. A substrate comprising a plurality of regions, wherein each region comprises
(a) a promoter primer that can be extended to form a transcribable template nucleic acid, and
(b) a first capture probe.

23. The substrate of claim 22 wherein the first capture probe of each region is non-extendable.

24. The substrate of claim 22 wherein the first capture probe and the promoter primer do not interact.

25. The substrate of claim 22 wherein the promoter primer cannot be extended

26. The substrate of claim 22 wherein the promoter primer and the first capture probe are both covalently coupled to the substrate.

27. The substrate of claim 22 wherein each region further comprises a second capture probe.

28. The substrate of claim 27 wherein the first and second capture probe can detect different cellular nucleic acids.

29. The substrate of claim 27 wherein the first and second capture probes recognize different regions of a common cellular nucleic acid, and the plurality of regions includes regions for different cellular nucleic acids.

30. A nucleic acid detection kit comprising
the substrate of claim 1 or 22; and
a detector nucleic acid.

31. The kit of claim 30 further comprising reagents for forming a transcribable template nucleic acid from the promoter primer.

32. The kit of claim 30 wherein the detector nucleic acid is labeled.

33. A device comprising:
(a) means for forming transcribable templates for a plurality of different nucleic acids,
(b) means for capturing specific nucleic acids, and
(c) means for supporting (a) and (b).

34. A method comprising:
contacting a sample to a substrate that comprises an immobilized promoter primer, wherein the promoter primer includes a sequence that forms at least one strand of a transcription recruitment sequence and an annealing sequence;
producing a transcribable nucleic acid template by extended the promoter primer, if the sample contains a sequence that can hybridize to the annealing sequence;
transcribing the transcribable nucleic to produce transcripts; and
evaluating interaction between transcripts and a capture probe without removing the transcripts from the substrate.

35. The method of claim 34 wherein the capture probe is immobilized on the substrate.

36. The method of claim 34 wherein the capture probe is immobilized on the substrate prior to contacting the sample to the substrate.

37. The method of claim 34 wherein the promoter primer is immobilized on the substrate prior to contacting the sample to the substrate.

38. The method of claim 34 wherein the evaluating comprises hybridizing detector probe(s) to the transcripts and washing detector probe(s) that are not attached to the substrate in a complex that comprises the capture probe and the transcript from the substrate.

39. The method of claim 34 wherein the sample includes less than 1 pg of the target nucleic acid.

40. A method of providing a substrate, the method comprising:
providing a substrate that comprises a plurality of regions; and
modifying the substrate so that each region comprises
(a) a promoter primer that can be extended to form a transcribable template nucleic acid, and
(b) a capture probe.

41. The method of claim 40 wherein the modifying comprises synthesizing the promoter primer and/or the capture probe on the substrate.

42. The method of claim 40 wherein the modifying comprises disposing the promoter primer and the capture probe on the substrate.

43. The method of claim 42 wherein the promoter primer is disposed in one subregion of the region, and the capture probe is disposed in another subregion of the region.

44. The method of claim 40 wherein the substrate comprises demarcations that physically separate the regions of the plurality from each other.
